(19) United States
(12) Patent Application Publication
(10) Pub. No.: US 2022/0154249 A1
(43) Pub. Date: May 19, 2022

(54) IMPROVED LIQUID BIOPSY USING SIZE SELECTION

(71) Applicant: Natera, Inc., San Carlos, CA (US)

(72) Inventors: Bernhard ZIMMERMANN, Manteca, CA (US); Ryan SWENERTON, Millbrae, CA (US); Fei LU, Fremont, CA (US); James STRAY, San Mateo, CA (US); Jason TONG, San Carlos, CA (US)

(73) Assignee: Natera, Inc., San Carlos, CA (US)

(21) Appl. No.: 17/600,789

(22) PCT Filed: Apr. 14, 2020
(86) PCT No.: PCT/US2020/028041

§ 371 Date: Oct. 1, 2021

Related U.S. Application Data
(60) Provisional application No. 62/833,915, filed on Apr. 15, 2019.

(51) Int. Cl. C12Q 1/6806; C12Q 1/686

(52) U.S. Cl. CPC C12Q 1/6806; C12Q 1/686

(57) ABSTRACT

Provided herein are improved methods of determining the sequences of cell-free DNA (cfDNA). The methods in certain embodiments are used for the analysis of circulating DNA in serum samples, such as circulating fetal DNA, circulating donor derived DNA, or circulating tumor DNA. In certain embodiments, the methods include selectively enriching trinucleosomal, dinucleosomal, mononucleosomal or sub-mononucleosomal DNA from the isolated cfDNA.

### CROSS REFERENCE TO RELATED APPLICATIONS

This application claims priority to U.S. Provisional Application No. 62/833,915 filed Apr. 15, 2019, which is hereby incorporated by reference in its entirety.

### BACKGROUND

Non-invasive and minimally invasive liquid biopsy tests utilize sample material collected from external secretions or by needle aspiration for analysis. The extracellular nuclear DNA present in the cell-free fraction of bodily fluids such as blood, plasma, serum, urine, saliva and other glandular secretions, cerebrospinal and peritoneal fluid, contain sufficient amounts of genomic sequences to support accurate detection of genetic anomalies that underlie many disorders that could otherwise be difficult or impossible to diagnosis outside of expensive medical biopsy procedures bearing substantial risk. In blood, the circulating cell free DNA (cfDNA) fraction represents a sampling of nucleic acid sequences shed into the blood from numerous sources which are deposited there as part of the normal physiological condition. The origin of a majority of cfDNA can be traced to either hematological processes or steady-state turnover of other tissues such as skin, muscle, and major organ systems. Of great clinical importance was the discovery that a significant and detectable fraction of cfDNA derives from exchange of fetal DNA crossing the placental boundary and from immune-mediated, apoptotic or necrotic cell lysis of tumor cells or cells infected by viruses, bacterium, or intracellular parasites. This makes plasma an extremely attractive specimen for molecular analytical tests and in particular, test that leverage the power of deep sequencing for diagnosis and detection.

The steady-state concentration of circulating cell free DNA (cfDNA) fluctuates in the ng/mL range, and reflects the net balance between release of fragmented chromatin into the bloodstream and the rate of clearance by nucleases, hepatic uptake and cell mediated engulfment. The key to liquid biopsy approaches which target cfDNA, is the ability to bind and purify sufficient quantities of the highly fragmented DNA from blood plasma collected by needle stick, typically from an arm vein. With respect to cancer monitoring, a problem is presented by the fact that an overwhelming majority of cfDNA in the biological sample comes from normal cells. Similarly, in the context of prenatal diagnosis, the overwhelming majority of cfDNA in the biological sample comes from maternal cells, and in the context of monitoring transplanted organs, most of the cfDNA in the biological sample comes from host cells. Thus, there remain a need for methods of enriching for cfDNA derived from a fetus, cancer cells, or a transplanted organ, for non-invasive prenatal testing, cancer monitoring, and transplant monitoring.

### SUMMARY OF THE INVENTION

The present disclosure provides a method of enriching for cfDNA coming from the target tissue to provide improved diagnostic methods based on liquid biopsy.

In one aspect, this disclosure provides a method for determining the sequences of cell-free DNA (cfDNA), comprising

(a) isolating cfDNA from a biological sample of a subject;

(b) optionally, ligating adaptors to the isolated cfDNA to obtain adaptor-ligated DNA, and/or amplifying the adaptor-ligated DNA to obtain amplified adaptor-ligated DNA;

(c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA;

(d) determining the sequences of the selectively enriched DNA.

In some embodiments, the biological sample is a blood, plasma, serum, or urine sample.

In some embodiments, step (b) of the method for determining the sequences of cell-free DNA (cfDNA) comprises ligating adaptors to the isolated cfDNA to obtain adaptor-ligated DNA, and step (c) comprises selectively enriching trinucleosomal, dinucleosomal, mononucleosomal or sub-mononucleosomal DNA from the adaptor-ligated DNA.

In some embodiments, step (b) of the method for determining the sequences of cell-free DNA (cfDNA) comprises ligating adaptors to the isolated cfDNA to obtain adaptor-ligated DNA and amplifying the adaptor-ligated DNA to obtain amplified adaptor-ligated DNA, and step (c) comprises selectively enriching trinucleosomal, dinucleosomal, mononucleosomal or sub-mononucleosomal DNA from the amplified adaptor-ligated DNA.

In some embodiments, step (c) of the method for determining the sequences of cell-free DNA (cfDNA) comprises performing size selection by gel electrophoresis, paramagnetic beads, spin column, salt precipitation, or biased amplification.

In some embodiments, step (d) of the method for determining the sequences of cell-free DNA (cfDNA) comprises performing a multiplex amplification reaction to amplify a plurality of polymorphic loci on the selectively enriched DNA in one reaction mixture.

In some embodiments, step (d) of the method for determining the sequences of cell-free DNA (cfDNA) comprises performing hybrid capture to select a plurality of polymorphic loci on the selectively enriched DNA.

In some embodiments, step (d) of the method for determining the sequences of cell-free DNA (cfDNA) comprises performing high-throughput sequencing.

In some embodiments, step (d) of the method for determining the sequences of cell-free DNA (cfDNA) comprises performing microarray analysis.

In some embodiments, step (d) of the method for determining the sequences of cell-free DNA (cfDNA) comprises performing qPCR or ddPCR analysis.

In some embodiments, step (c) further comprises performing hybrid capture to select a plurality of polymorphic loci on the isolated cfDNA, the adaptor-ligated DNA, and/or amplified adaptor-ligated DNA prior to selectively enriching trinucleosomal, dinucleosomal, mononucleosomal or sub-mononucleosomal DNA.

In some embodiments, step (c) comprises selectively enriching dinucleosomal, mononucleosomal or sub-mononucleosomal DNA from the isolated cfDNA, the adaptor-ligated DNA or the amplified adaptor-ligated DNA to obtain selectively enriched DNA. In some embodiments, step (c) comprises selectively enriching mononucleosomal or sub-mononucleosomal DNA from the isolated cfDNA, the adaptor-ligated DNA or the amplified adaptor-ligated DNA to obtain selectively enriched DNA. In some embodiments, wherein step (c) comprises selectively enriching sub-mononucleosomal DNA from the isolated cfDNA, the adaptor-ligated DNA or the amplified adaptor-ligated DNA to obtain selectively enriched DNA.

Non-Invasive Pre-Natal Testing

In another aspect, this disclosure provides a method for non-invasive prenatal testing, comprising

(a) isolating cfDNA from a biological sample of a pregnant woman, wherein the isolated cfDNA comprises a mixture of fetal cfDNA and maternal cfDNA;

(b) optionally, ligating adaptors to the isolated cfDNA to obtain adaptor-ligated DNA, and/or amplifying the adaptor-ligated DNA to obtain amplified adaptor-ligated DNA;

(c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of fetal cfDNA;

(d) performing a multiplex amplification reaction to amplify at least 100 polymorphic loci on the selectively enriched DNA in one reaction mixture; and

(e) determining the sequences of the selectively enriched DNA.

In some embodiments, the fraction of fetal cfDNA is increased by at least 10%, at least 20%, at least 30%, at least 50%, at least 100%, at least 200%, or at least 300%, in the selectively enriched DNA compared to the isolated cfDNA.

In some embodiments, the method for non-invasive prenatal testing further comprises determining the presence of at least one fetal chromosomal abnormality based on the sequences of the selectively enriched DNA.

In some embodiments, the method for non-invasive prenatal testing further comprises that the fetal chromosomal abnormality comprises single nucleotide variant (SNV), copy number variation (CNV), and/or chromosomal rearrangement.

In some embodiments, the biological sample is a blood, plasma, serum, or urine sample.

In some embodiments, step (b) comprises ligating adaptors to the isolated cfDNA to obtain adaptor-ligated DNA, and step (c) comprises selectively enriching trinucleosomal, dinucleosomal, mononucleosomal or sub-mononucleosomal DNA from the adaptor-ligated DNA.

In some embodiments, step (b) comprises ligating adaptors to the isolated cfDNA to obtain adaptor-ligated DNA and amplifying the adaptor-ligated DNA to obtain amplified adaptor-ligated DNA, and step (c) comprises selectively enriching trinucleosomal, dinucleosomal, mononucleosomal or sub-mononucleosomal DNA from the amplified adaptor-ligated DNA.

In some embodiments, step (c) comprises performing size selection by gel electrophoresis, paramagnetic beads, spin column, salt precipitation, or biased amplification.

In some embodiments, step (d) comprises amplifying at least 200, at least 500, at least 1,000, at least 2,000, at least 5,000, or at least 10,000 polymorphic loci on the selectively enriched DNA in one reaction mixture.

In some embodiments, step (e) comprises performing high-throughput sequencing, microarray, qPCR or ddPCR analysis.

In some embodiments, step (c) further comprises performing hybrid capture to select a plurality of polymorphic loci on the isolated cfDNA, the adaptor-ligated DNA, and/or amplified adaptor-ligated DNA prior to selectively enriching trinucleosomal, dinucleosomal, mononucleosomal or sub-mononucleosomal DNA.

In some embodiments, step (c) comprises selectively enriching dinucleosomal, mononucleosomal or sub-mononucleosomal DNA from the isolated cfDNA, the adaptor-ligated DNA or the amplified adaptor-ligated DNA to obtain selectively enriched DNA. In some embodiments, step (c) comprises selectively enriching mononucleosomal or sub-mononucleosomal DNA from the isolated cfDNA, the adaptor-ligated DNA or the amplified adaptor-ligated DNA to obtain selectively enriched DNA. In some embodiments, wherein step (c) comprises selectively enriching sub-mononucleosomal DNA from the isolated cfDNA, the adaptor-ligated DNA or the amplified adaptor-ligated DNA to obtain selectively enriched DNA.

Transplant Monitoring

In one aspect, the present disclosure provides a method for monitoring transplant rejection, comprising

(a) isolating cfDNA from a biological sample of a transplant recipient, wherein the isolated cfDNA comprises a mixture of donor-derived cfDNA and recipient cfDNA;

(b) optionally, ligating adaptors to the isolated cfDNA to obtain adaptor-ligated DNA, and/or amplifying the adaptor-ligated DNA to obtain amplified adaptor-ligated DNA;

(c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of donor-derived cfDNA;

(d) performing a multiplex amplification reaction to amplify at least 100 polymorphic loci on the selectively enriched DNA in one reaction mixture; and

(e) determining the sequences of the selectively enriched DNA.

In some embodiments, the fraction of donor-derived cfDNA is increased by at least 10%, at least 20%, at least 30%, at least 50%, at least 100%, at least 200%, or at least 300%, in the selectively enriched DNA compared to the isolated cfDNA.

In some embodiments, the method further comprises quantifying the amount of donor-derived cfDNA.

In some embodiments, the method further comprises determining the likelihood of transplant rejection based on the amount of donor-derived cfDNA.

In some embodiments, the biological sample is a blood, plasma, serum, or urine sample.

In some embodiments, step (b) comprises ligating adaptors to the isolated cfDNA to obtain adaptor-ligated DNA, and step (c) comprises selectively enriching trinucleosomal, dinucleosomal, mononucleosomal or sub-mononucleosomal DNA from the adaptor-ligated DNA.

In some embodiments, step (b) comprises ligating adaptors to the isolated cfDNA to obtain adaptor-ligated DNA and amplifying the adaptor-ligated DNA to obtain amplified adaptor-ligated DNA, and step (c) comprises selectively enriching trinucleosomal, dinucleosomal, mononucleosomal or sub-mononucleosomal DNA from the amplified adaptor-ligated DNA.

In some embodiments, step (c) comprises performing size selection by gel electrophoresis, paramagnetic beads, spin column, salt precipitation, or biased amplification.

In some embodiments, step (d) comprises amplifying at least 200, at least 500, at least 1,000, at least 2,000, at least 5,000, or at least 10,000 polymorphic loci on the selectively enriched DNA in one reaction mixture.

In some embodiments, step (e) comprises performing high-throughput sequencing, microarray, qPCR or ddPCR analysis.

In some embodiments, the method comprises longitudinally collecting one or more biological samples from the transplant recipient after transplantation, and repeating steps (a)-(e) for each biological samples longitudinally collected, in order to monitor transplant rejection.

In some embodiments, step (c) further comprises performing hybrid capture to select a plurality of polymorphic loci on the isolated cfDNA, the adaptor-ligated DNA, and/or amplified adaptor-ligated DNA prior to selectively enriching trinucleosomal, dinucleosomal, mononucleosomal or sub-mononucleosomal DNA.

In some embodiments, step (c) comprises selectively enriching dinucleosomal, mononucleosomal or sub-mononucleosomal DNA from the isolated cfDNA, the adaptor-ligated DNA or the amplified adaptor-ligated DNA to obtain selectively enriched DNA. In some embodiments, step (c) comprises selectively enriching mononucleosomal or sub-mononucleosomal DNA from the isolated cfDNA, the adaptor-ligated DNA or the amplified adaptor-ligated DNA to obtain selectively enriched DNA. In some embodiments, wherein step (c) comprises selectively enriching sub-mononucleosomal DNA from the isolated cfDNA, the adaptor-ligated DNA or the amplified adaptor-ligated DNA to obtain selectively enriched DNA.

Cancer Monitoring

In another aspect, the present disclosure provides a method for monitoring relapse or metastasis of cancer, comprising

(a) isolating cfDNA from a biological sample of a subject diagnosed with cancer;

(b) optionally, ligating adaptors to the isolated cfDNA to obtain adaptor-ligated DNA, and/or amplifying the adaptor-ligated DNA to obtain amplified adaptor-ligated DNA;

(c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of circulating tumor DNA (ctDNA);

(d) performing a multiplex amplification reaction to amplify a plurality of patient-specific somatic mutations on the selectively enriched DNA in one reaction mixture, wherein the patient-specific somatic mutations are identified in a tumor sample of the subject; and

(e) determining the sequences of the selectively enriched DNA.

In another aspect, the present disclosure provides a method for monitoring relapse or metastasis of cancer, comprising

(a) isolating cfDNA from a biological sample of a subject diagnosed with cancer;

(b) optionally, ligating adaptors to the isolated cfDNA to obtain adaptor-ligated DNA, and/or amplifying the adaptor-ligated DNA to obtain amplified adaptor-ligated DNA;

(c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of circulating tumor DNA (ctDNA);

(d) enriching the selectively enriched DNA by hybrid capture for target regions each comprising at least one of a plurality of patient-specific somatic mutations, wherein the patient-specific somatic mutations are identified in a tumor sample of the subject; and

(e) determining the sequences of the selectively enriched DNA.

In another aspect, the present disclosure provides a method for monitoring relapse or metastasis of cancer, comprising

(a) isolating cfDNA from a biological sample of a subject diagnosed with cancer;

(b) optionally, ligating adaptors to the isolated cfDNA to obtain adaptor-ligated DNA, and/or amplifying the adaptor-ligated DNA to obtain amplified adaptor-ligated DNA;

(c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of circulating tumor DNA (ctDNA); and

(d) determining the sequences of the selectively enriched DNA by shotgun sequencing.

In some embodiments, the fraction of ctDNA is increased by at least 10%, at least 20%, at least 30%, at least 50%, at least 100%, at least 200%, or at least 300%, in the selectively enriched DNA compared to the isolated cfDNA.

In some embodiments, step (d) comprises amplifying at least 4, or at least 8, or at least 16, or at least 24, or at least 32, or at most 128, or at most 64, or at most 48, patient-specific somatic mutations on the selectively enriched DNA in one reaction mixture.

In some embodiments, the detection of two or more, three or more, four or more, or five or more patient-specific somatic mutations in the selectively enriched DNA is indicative of relapse or metastasis of cancer.

In some embodiments, the patient-specific somatic mutations comprise single nucleotide variant (SNV), copy number variation (CNV), and/or chromosomal rearrangement.

In some embodiments, the biological sample is a blood, plasma, serum, or urine sample.

In some embodiments, step (b) comprises ligating adaptors to the isolated cfDNA to obtain adaptor-ligated DNA, and step (c) comprises selectively enriching trinucleosomal, dinucleosomal, mononucleosomal or sub-mononucleosomal DNA from the adaptor-ligated DNA.

In some embodiments, step (b) comprises ligating adaptors to the isolated cfDNA to obtain adaptor-ligated DNA and amplifying the adaptor-ligated DNA to obtain amplified adaptor-ligated DNA, and step (c) comprises selectively enriching trinucleosomal, dinucleosomal, mononucleosomal or sub-mononucleosomal DNA from the amplified adaptor-ligated DNA.

In some embodiments, step (c) comprises performing size selection by gel electrophoresis, paramagnetic beads, spin column, salt precipitation, or biased amplification.

In some embodiments, step (e) comprises performing high-throughput sequencing, microarray, qPCR or ddPCR analysis.

In some of embodiments, the method comprises longitudinally collecting one or more biological samples from the subject after the patient has been treated with surgery, first-line chemotherapy, and/or adjuvant therapy, and repeating steps (a)-(e) for each biological samples longitudinally collected, in order to monitor cancer relapse and/or metastasis.

In some embodiments, step (c) further comprises performing hybrid capture to select a plurality of polymorphic loci on the isolated cfDNA, the adaptor-ligated DNA, and/or amplified adaptor-ligated DNA prior to selectively enriching trinucleosomal, dinucleosomal, mononucleosomal or sub-mononucleosomal DNA.

In some embodiments, step (c) comprises selectively enriching dinucleosomal, mononucleosomal or sub-mononucleosomal DNA from the isolated cfDNA, the adaptor-ligated DNA or the amplified adaptor-ligated DNA to obtain selectively enriched DNA. In some embodiments, step (c) comprises selectively enriching mononucleosomal or sub-mononucleosomal DNA from the isolated cfDNA, the adaptor-ligated DNA or the amplified adaptor-ligated DNA to obtain selectively enriched DNA. In some embodiments, wherein step (c) comprises selectively enriching sub-mononucleosomal DNA from the isolated cfDNA, the adaptor-ligated DNA or the amplified adaptor-ligated DNA to obtain selectively enriched DNA.

### BRIEF DESCRIPTION OF THE DRAWINGS

FIG. 1 is a diagram showing a workflow of trinucleosomal, dinucleosomal, mononucleosomal or submononucleosomal size selection on amplified library based on various size selection methods.

FIG. 2 is a diagram showing a workflow of size selection through biased library amplification PCR.

FIG. 3 depicts graphs showing the size distribution of maternal and fetal cell-free DNA (cfDNA). The graphs show that fetal cfDNA has a size peak at 143 bp and maternal cfDNA has a size peak at 166 bp.

FIG. 4 depicts a diagram showing the overall non-invasive prenatal testing (NIPT) workflow with fetal enrichment by size selection. The library re-amplification PCR reaction is optional.

FIG. 5 is a graph comparing child fraction estimate (CFE) before (light gray) and post size selection (dark grey) of 16 low risk samples and 4 confirmed Trisomy 21 samples. The samples were shown to have 2 to 5 fold (3 fold on average) fetal enrichment consistently. All samples were shown to have more than 8% CFE post size selection as indicated by the horizontal line cutoff at 8%.

FIG. 6 is a graph showing child fraction estimate (CFE) fold increase (y-axis) as a function of CFE before size selection (x-axis).

FIG. 7 is a graph showing examples of the size distribution of 2 cfDNA samples pre-size selection (solid arrow on the right side) and post-size selection (dotted arrow on the left side).

FIG. 8 is a graph showing the child fraction estimate (CFE) increase from pre-size selection to post-size selection of 16 healthy and 4 confirmed Trisomy 21 pregnancy samples.

FIG. 9 is a diagram showing a workflow of size selection for mononucleosomal DNA or subfraction of mononucleosomal DNA applied post hybrid capture or other pull-down methods.

### DETAILED DESCRIPTION

Reference will now be made in detail to some specific embodiments of the invention contemplated by the inventors for carrying out the invention. Certain examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

### Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

This disclosure provides methods for improving the confidence and accuracy of determining the sequences of cfDNA. In one aspect, this disclosure relates to a method of determining the sequences of cfDNA comprising (a) isolating cfDNA from a biological sample of a subject; (b) optionally, ligating adaptors to the isolated cfDNA to obtain adaptor-ligated DNA, and/or amplifying the adaptor-ligated DNA to obtain amplified adaptor-ligated DNA; (d) determining the sequences of the selectively enriched DNA. In some embodiments, this disclosure relates to a method of determining the sequences of cfDNA comprising (a) isolating cfDNA from a biological sample of a subject; (b) ligating adaptors to the isolated cfDNA to obtain adaptor-ligated DNA, and (c) selectively enriching trinucleosomal, dinucleosomal, mononucleosomal or sub-mononucleosomal DNA from the adaptor-ligated DNA. In some embodiments, this disclosure relates to a method of determining the sequences of cell-free DNA (cfDNA) comprising (a) isolating cfDNA from a biological sample of a subject; (b) ligating adaptors to the isolated cfDNA to obtain adaptor-ligated DNA and amplifying the adaptor-ligated DNA to obtain amplified adaptor-ligated DNA, and (c) selectively enriching trinucleosomal, dinucleosomal, mononucleosomal or sub-mononucleosomal DNA from the amplified adaptor-ligated DNA.

As used herein, the term “cell-free DNA” or “cfDNA” refers to DNA that is free-floating in biological samples. In some embodiments, the biological sample is a blood, plasma, serum, or urine sample. In some embodiments, the biological sample is from a pregnant mother. In some embodiments, the isolated cfDNA is a mixture of fetal and maternal cfDNA.

The term “single nucleotide polymorphism (SNP)” refers to a single nucleotide that may differ between the genomes of two members of the same species. The usage of the term should not imply any limit on the frequency with which each variant occurs.

The term “sequence” refers to a DNA sequence or a genetic sequence. It may refer to the primary, physical structure of the DNA molecule or strand in an individual. It may refer to the sequence of nucleotides found in that DNA molecule, or the complementary strand to the DNA molecule. It may refer to the information contained in the DNA molecule as its representation in silico.

The term “locus” refers to a particular region of interest on the DNA of an individual, which may refer to a SNP, the site of a possible insertion or deletion, or the site of some other relevant genetic variation. Disease-linked SNPs may also refer to disease-linked loci.

The term “polymorphic allele” or “polymorphic locus” refers to an allele or locus where the genotype varies between individuals within a given species. Some examples of polymorphic alleles include single nucleotide polymorphisms, short tandem repeats, deletions, duplications, and inversions.

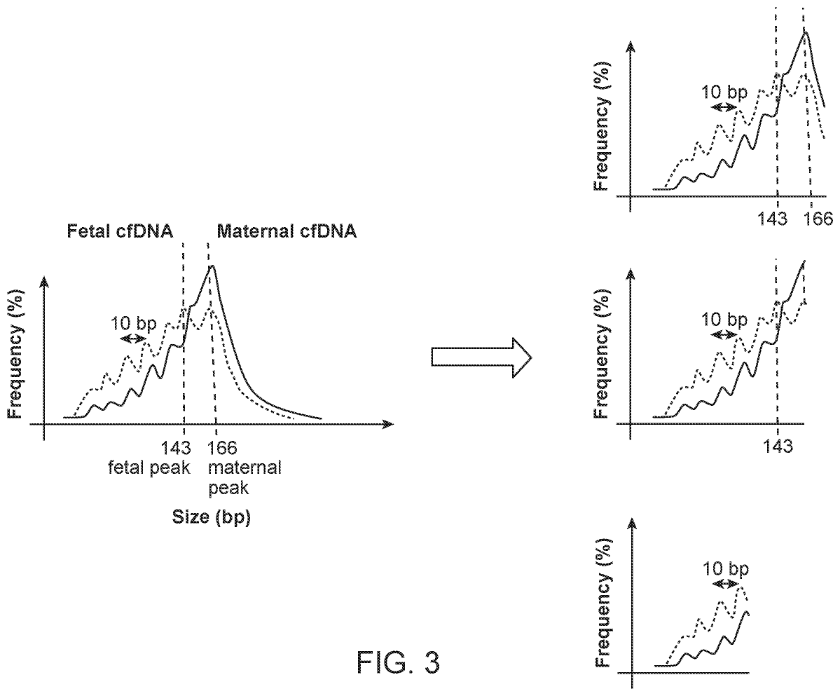

The term “isolating” as used herein refers to a physical separation of the target genetic material from other contaminating genetic material or biological material. It may also refer to a partial isolation, where the target of isolation is separated from some or most, but not all of the contaminating material. It has been shown that cfDNA may exist as nucleosomal complexes with the DNA tightly wrapped around histones. Mononucleosomal complexes consists of about 130 to about 170 bp of DNA wrapped around a single nucleosome. The term “trinucleosomal” refers to a fragment of chromosomal DNA containing three nucleosomes. The term “dinucleosomal” refers to a fragment of chromosomal DNA containing two nucleosomes. The term “mononucleosomal” refers to a fragment of chromosomal DNA containing a single nucleosome. The term “sub-mononucleosomal” refers to a fragment of chromosomal DNA having smaller molecular size than about 130 bp that would be expected to derive from a complete nucleosome. cfDNA may also exist integrated in lipid vesicles such as exosomes. FIG. 3 shows the size distribution of fetal and maternal cfDNA. Fetal cfDNA has a peak size at 143 bp and maternal cfDNA has a peak size at 164 bp. Accordingly, the methods of isolating the cfDNA must ensure preservation of the cfDNA fragments have molecular size below 200 bp.

Chromosomal DNA consists of DNA wrapped around a complex of histone proteins that forms a nucleosome. The nucleosome protects the DNA so that fragmented chromosomal DNA are often found as multiples of nucleosomes.

Many methods known by a person of ordinary skill in the art may be used to isolate cell-free DNA from a biological sample. Such methods include but are not limited to organic liquid phase extraction utilizing phenol and phenol-chloroform mixtures to disintegrate nucleoprotein complexes and sequester proteins and lipids into the organic phase while partitioning the highly hydrophilic DNA and RNA into the aqueous phase in very pure form. Other methods include using agarose hydrogels such as those described in E. M. Southern (J. Mol. Biol. (1975) 94:51-70) and Vogelstein and Gillespie (PNAS, USA (1979)76:615-619), incorporated herein in their entirety. Another method is to capture DNA on a solid phase material as described in Boom et al. (J Clin Micro. (1990) 28(3):495-503), incorporated herein in its entirety. Methods for DNA isolation in general can be found in Sambrook J, Russel D W (2001). Molecular Cloning: A Laboratory Manual 3rd Ed. Cold Spring Harbor Laboratory Press. Cold Spring Harbor, N.Y., incorporated herein.

Further methods described in detail below can be used to enrich for DNA fragments within specific molecular size ranges. It is a discovery of the disclosure herein, that enriching for smaller cfDNA fragments greatly improves the accuracy and confidence of cfDNA based diagnostic tests. As shown in Example 1 herein, enriching for adaptor ligated cfDNA derived from blood samples from pregnant women in the molecular weight range from 100 to 237 bp (cfDNA size range without the ligated adaptor can be 33-170 bp), resulted in a 2-5 folds (3 folds on average) enrichment of fetal cfDNA.

### Size Selection/Exclusion Methods

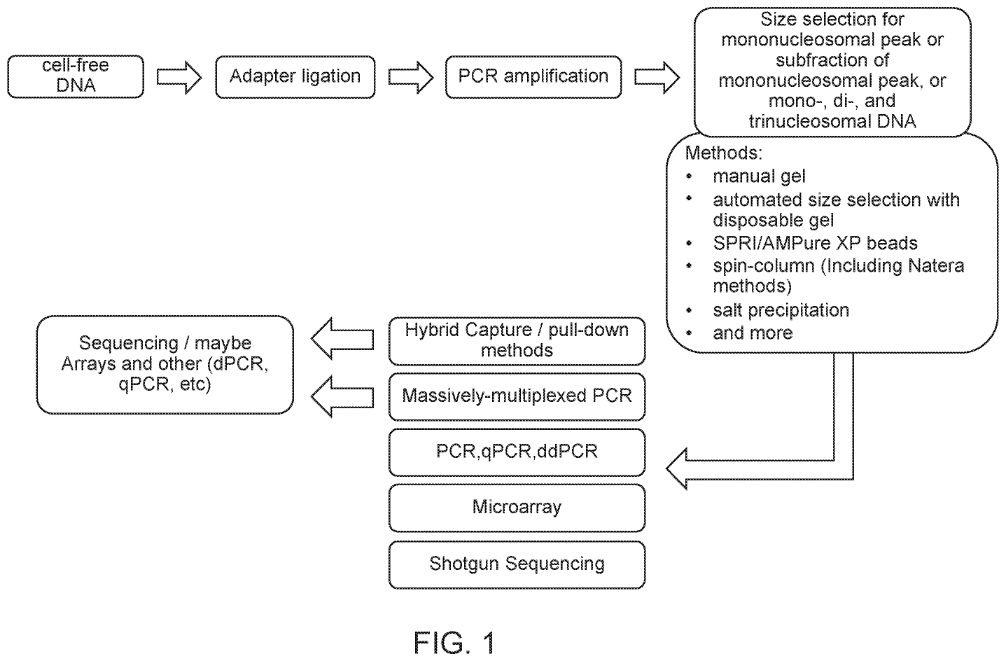

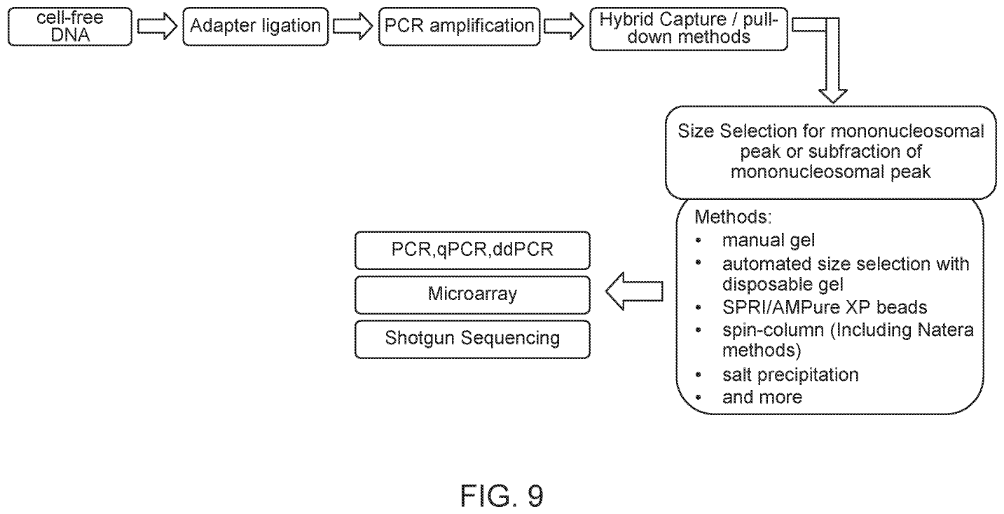

This disclosure relates to methods comprising performing size selection by gel electrophoresis, paramagnetic beads, spin column, salt precipitation, or biased amplification. FIGS. 1, 2, and 9 show example workflows of the methods.

In some embodiments, the size exclusion step of the methods disclosed herein is performed by using gel electrophoresis to separate the cfDNA samples according to size and selecting a determined size range. Gel electrophoresis is an art-recognized method for separating DNA molecules based on their size by applying an electric field to a gel, such as an agarose gel, upon which DNA molecules will move through the gel towards the positively charged anode. The size of the DNA molecules will determine the speed by which the DNA molecule migrate through the gel. A standard mixture of DNA molecules with predetermined sizes can be applied to the gel to identify the size of the DNA. The DNA molecules of desired size can then be extracted and purified by using well-known techniques such as those disclosed in Sambrook J, Russel D W (2001). Molecular Cloning: A Laboratory Manual 3rd Ed. Cold Spring Harbor Laboratory Press. Cold Spring Harbor, N.Y., incorporated. In some embodiments, the size selection is performed on an automated high-throughput gel electrophoresis system such as Pippin or Costal Genomics systems.

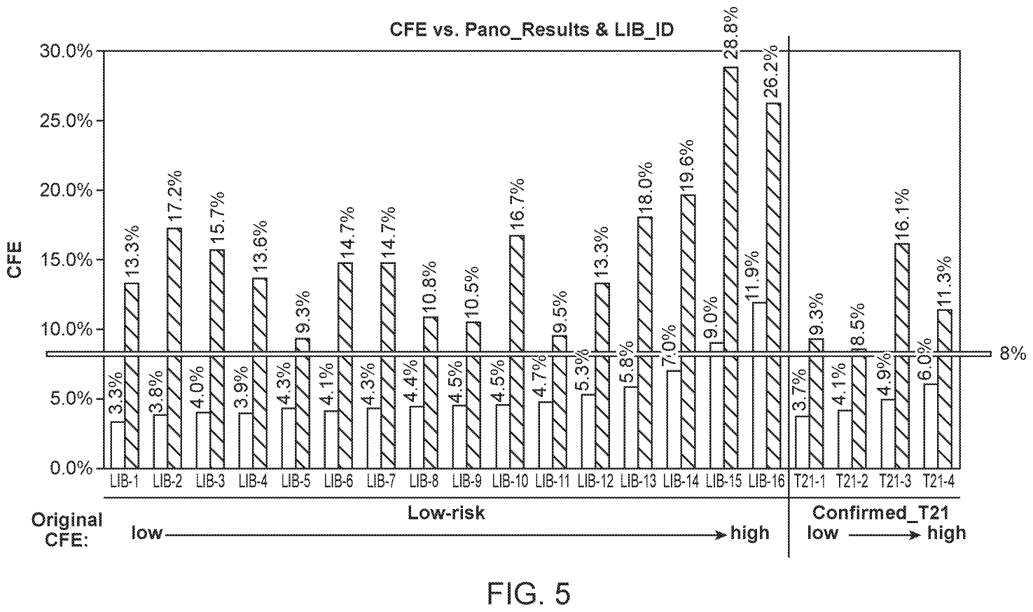

In one illustrative example, the method disclosed herein used gel electrophoresis to enrich for DNA fragments in the range 100 to 270 bp as further explained in Example 1. This size exclusion step was performed on 20 samples and resulted in a 2 to 5 folds enrichment of % child fraction estimate as shown in FIG. 5.

In some embodiments, the size exclusion step of the methods disclosed herein is performed by using paramagnetic beads. The use of paramagnetic beads for size selection of DNA fragments is described in DeAngelis et al., Solid-Phase Reversible Immobilization for the Isolation of PCR Products, Nucleic Acid Research, November 23(22): 4742-3 (1995), incorporated herein. In brief, this method is based on that DNA fragment size affects the total charge per molecule with larger DNAs having larger charges, which promotes their electrostatic interaction with the beads and displaces smaller DNA fragments. Thus, by manipulating the composition of the buffer solution used to mix beads and DNA, the beads can be made to bind DNA within specific size ranges. The most famous and highly applied approach is Solid Phase Reversible Isolation (SPRI) selection which utilizes carboxyl coated paramagnetic beads in the presence of high salt and the crowding agent polyethylene glycol (PEG), to promote controlled adsorption, configure to bind DNA molecules within a certain molecular weight ranges by varying PEG concentrations. DNA molecules of differing length can be partitioned by subjecting source DNA to various binding and elution schemes in the presence of different amounts of PEG. In some embodiments, AMPURE™ beads are used for the size exclusion step.

In some embodiments, the size exclusion step of the methods disclosed herein is performed by using spin columns. A spin column contains material that will absorb molecules based on the size of the molecules. The spin column material contains pores of defined sizes and molecules with a size above a cutoff size determined by the pore size will not enter the pores, and are eluted with the column's void volume. Different types of column material can be chosen to achieve absorption or exclusion of DNA molecules within various size ranges. In some embodiments, the spin column material comprises siliceous materials, silica gel, glass, glass fiber, zeolite, aluminum oxide, titanium dioxide, zirconium dioxide, kaolin, gelatinous silica, magnetic particles, ceramics, polymeric supporting materials, or a combination thereof. In a particular embodiment, the spin column material comprises glass fiber.

In some embodiments, spin columns may be used for size exclusion by using different binding buffers configured to provide low or high stringency binding conditions when applying the DNA samples to the spin column, as described in PCT patent application No. PCT/US2019/18274 filed on Feb. 15, 2019, which is incorporated herein by reference in its entirety. Under low stringency binding conditions, the spin column material be configured to restrict binding of DNA fragments of low molecular weights, whereas high stringency binding conditions will configure the spin column to facilitate binding of DNA fragments with low molecular weights.

In some embodiments, the low and/or high stringency binding buffer comprises a nitrile compound selected from acetonitrile (ACN), propionitrile (PCN), butyronitrile (BCN), isobutylnitrile (IBCN), or a combination thereof. The first and/or second binding buffer can comprise, for example, about 15% to about 35%, or about 20% to about 30%, or about 25% of the nitrile compound (e.g., ACN).

In some embodiments, the low and/or high stringency binding buffer comprises a chaotropic compound selected from GnCl, urea, thiourea, guanidine thiocyanate, NaI, guanidine isothiocyanate, D-/L-arginine, a perchlorate or perchlorate salt of Li+, Na+, K+, or a combination thereof. The low and/or high stringency binding buffer can comprise, for example, about 5 M to about 8 M, or about 5.6 M to about 7.2 M, or about 6 M of the chaotropic compound (e.g., GnCl).

The binding buffers may also comprise an alcohol, a chelating agent, and a detergent. In some embodiments, the alcohol is propanol. In some embodiments, the chelating compound comprises ethylenediaminetetraccetic (EDTA), ethyleneglycol-bis(2-aminoethylether)-N,N,N′,N′-tetraacetic acid (EGTA), citric acid, N,N,N′,N′-Tetrakis(2-pyridylmethyl)ethylenediamine (TPEN), 2,2′-Bipyridyl, deferoxamine methanesulfonate salt (DFOM), 2,3-Dihydroxybutanedioic acid (tartaric acid), or a combination thereof. In some embodiments, the detergent may be Triton X-100, Tween 20, N-lauroyl sarcosine, sodium dodecylsulfate (SDS), dodecyldimethylphosphine oxide, sorbitan monopalmitate, decylhexaglycol, 4-nonylphenyl-polyethylene glycol, or a combination thereof. In a particular embodiment, the detergent is Triton X-100.

In some embodiments, the size exclusion step of the methods disclosed herein is performed by using salt precipitation. Larger DNA molecules will precipitate at lower salt concentrations than smaller DNA molecules. By varying the concentration of salt in the precipitation buffer, DNA molecules in different size ranges can be separated.

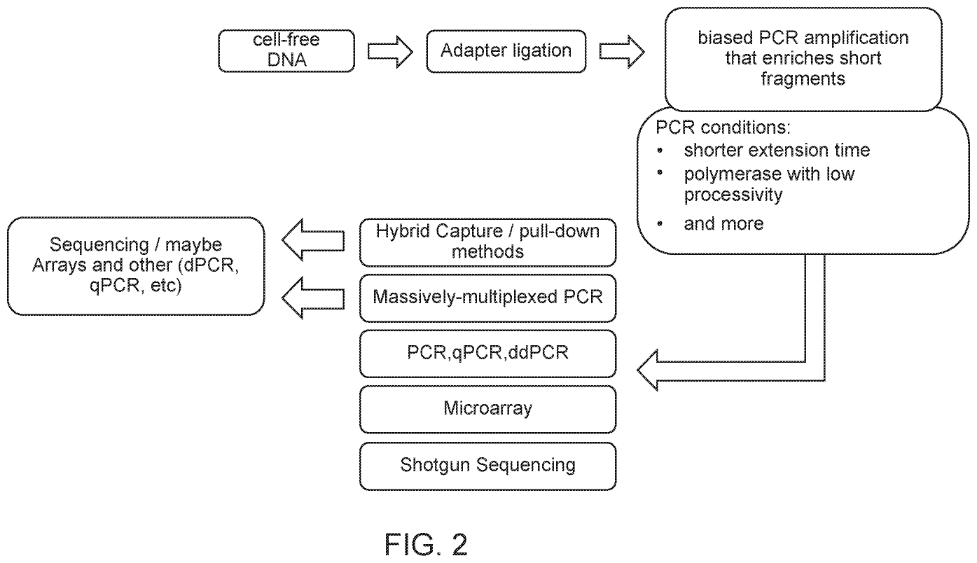

In some embodiments, the size exclusion step is performed by biased PCR. FIG. 2 shows a workflow of a method using biased library PCR amplification to enrich for shorter DNA molecules. In some embodiments, biased PCR can enrich for shorter DNA molecules by using shorter time for DNA extension in the PCR cycle protocol. If desired, the extension step of the PCR amplification may be limited from a time standpoint to reduce amplification from fragments longer than 200 nucleotides, 300 nucleotides, 400 nucleotides, 500 nucleotides or 1,000 nucleotides. This may result in the enrichment of fragmented or shorter DNA (such as fetal DNA or DNA from cancer cells that have undergone apoptosis or necrosis) and improvement of test performance.

In some embodiments, biased PCR can enrich for shorter DNA molecules by using a polymerase with low processivity. FIG. 2 outlines an illustrative method of evaluating cfDNA that incorporated biased PCR to enrich for shorter DNA molecules.

### Methods of Determining the Sequences of the Selectively Enriched DNA

Multiplex PCR Methods

In some embodiments, the method comprises performing a multiplex amplification reaction to amplify a plurality of polymorphic loci on the selectively enriched DNA in one reaction mixture before determining the sequences of the selectively enriched DNA.

In certain illustrative embodiments, the nucleic acid sequence data is generated by performing high throughput DNA sequencing of a plurality of copies of a series of amplicons generated using a multiplex amplification reaction, wherein each amplicon of the series of amplicons spans at least one polymorphic loci of the set of polymorphic loci and wherein each of the polymeric loci of the set is amplified. For example, in these embodiments a multiplex PCR to amplify amplicons across the 1,000 to 50,000 polymeric loci and the 100 to 1000 single nucleotide variant sites may be performed. This multiplex reaction can be set up as a single reaction or as pools of different subset multiplex reactions. The multiplex reaction methods provided herein, such as the massive multiplex PCR disclosed herein provide an exemplary process for carrying out the amplification reaction to help attain improved multiplexing and therefore, sensitivity levels.

In some embodiments, amplification is performed using direct multiplexed PCR, sequential PCR, nested PCR, doubly nested PCR, one-and-a-half sided nested PCR, fully nested PCR, one sided fully nested PCR, one-sided nested PCR, hemi-nested PCR, hemi-nested PCR, triply hemi-nested PCR, semi-nested PCR, one sided semi-nested PCR, reverse semi-nested PCR method, or one-sided PCR, which are described in U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, and U.S. Ser. No. 61/994,791, filed May 16, 2014, which are hereby incorporated by reference in their entirety.

In some embodiments, multiplex PCR is used. In some embodiments, the method of amplifying target loci in a nucleic acid sample involves (i) contacting the nucleic acid sample with a library of primers that simultaneously hybridize to least 100; 200; 500; 750; 1,000; 2,000; 5,000; 7,500; 10,000; 20,000; 25,000; 30,000; 40,000; 50,000; 75,000; or 100,000 different target loci to produce a single reaction mixture; and (ii) subjecting the reaction mixture to primer extension reaction conditions (such as PCR conditions) to produce amplified products that include target amplicons. In some embodiments, at least 50, 60, 70, 80, 90, 95, 96, 97, 98, 99, or 99.5% of the targeted loci are amplified. In various embodiments, less than 60, 50, 40, 30, 20, 10, 5, 4, 3, 2, 1, 0.5, 0.25, 0.1, or 0.05% of the amplified products are primer dimers. In some embodiments, the primers are in solution (such as being dissolved in the liquid phase rather than in a solid phase). In some embodiments, the primers are in solution and are not immobilized on a solid support. In some embodiments, the primers are not part of a microarray.

In certain embodiments, the multiplex amplification reaction is performed under limiting primer conditions for at least ½ of the reactions. In some embodiments, limiting primer concentrations are used in 1/10, ⅕, ¼, ⅓, ½, or all of the reactions of the multiplex reaction. Provided herein are factors to consider to achieve limiting primer conditions in an amplification reaction such as PCR.

In certain embodiments, methods provided herein detect ploidy for multiple chromosomal segments across multiple chromosomes. Accordingly, the chromosomal ploidy in these embodiments is determined for a set of chromosome segments in the sample. For these embodiments, higher multiplex amplification reactions are needed. Accordingly, for these embodiments the multiplex amplification reaction can include, for example, between 2,500 and 50,000 multiplex reactions. In certain embodiments, the following ranges of multiplex reactions are performed: between 100, 200, 250, 500, 1000, 2500, 5000, 10,000, 20,000, 25000, 50000 on the low end of the range and between 200, 250, 500, 1000, 2500, 5000, 10,000, 20,000, 25000, 50000, and 100,000 on the high end of the range.

In an embodiment, a multiplex PCR assay is designed to amplify potentially heterozygous SNP or other polymorphic or non-polymorphic loci on one or more chromosomes and these assays are used in a single reaction to amplify DNA. The number of PCR assays may be between 50 and 200 PCR assays, between 200 and 1,000 PCR assays, between 1,000 and 5,000 PCR assays, or between 5,000 and 20,000 PCR assays (50 to 200-plex, 200 to 1,000-plex, 1,000 to 5,000-plex, 5,000 to 20,000-plex, more than 20,000-plex respectively). In an embodiment, a multiplex pool of about 10,000 PCR assays (10,000-plex) are designed to amplify potentially heterozygous SNP loci on chromosomes X, Y, 13, 18, and 21 and 1 or 2 and these assays are used in a single reaction to amplify cfDNA obtained from a material plasma sample, chorion villus samples, amniocentesis samples, single or a small number of cells, other bodily fluids or tissues, cancers, or other genetic matter. The SNP frequencies of each locus may be determined by clonal or some other method of sequencing of the amplicons. Statistical analysis of the allele frequency distributions or ratios of all assays may be used to determine if the sample contains a trisomy of one or more of the chromosomes included in the test. In another embodiment the original cfDNA samples is split into two samples and parallel 5,000-plex assays are performed. In another embodiment the original cfDNA samples is split into n samples and parallel (˜10,000/n)-plex assays are performed where n is between 2 and 12, or between 12 and 24, or between 24 and 48, or between 48 and 96.

Bioinformatics methods are used to analyze the genetic data obtained from multiplex PCR. The bioinformatics methods useful and relevant to the methods disclosed herein can be found in U.S. Patent Publication No. 20180025109, incorporated by reference herein.

Hybrid Capture Methods

In some embodiments, the method comprises performing hybrid capture to select a plurality of polymorphic loci on the selectively enriched DNA before determining the sequences of the selectively enriched DNA.

In some embodiments, step (c) further comprises performing hybrid capture to select a plurality of polymorphic loci on the isolated cfDNA, the adaptor-ligated DNA, and/or amplified adaptor-ligated DNA prior to selectively enriching trinucleosomal, dinucleosomal, mononucleosomal or sub-mononucleosomal DNA.

In some embodiments, preferentially enriching the DNA at the plurality of polymorphic loci includes obtaining a plurality of hybrid capture probes that target the polymorphic loci, hybridizing the hybrid capture probes to the DNA in the sample and physically removing some or all of the unhybridized DNA from the first sample of DNA.

In some embodiments, the hybrid capture probes are designed to hybridize to a region that is flanking but not overlapping the polymorphic site. In some embodiments, the hybrid capture probes are designed to hybridize to a region that is flanking but not overlapping the polymorphic site, and where the length of the flanking capture probe may be selected from the group consisting of less than about 120 bases, less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases. In some embodiments, the hybrid capture probes are designed to hybridize to a region that overlaps the polymorphic site, and where the plurality of hybrid capture probes comprise at least two hybrid capture probes for each polymorphic loci, and where each hybrid capture probe is designed to be complementary to a different allele at that polymorphic locus.

High-Throughput Sequencing

In some embodiments, the sequences of the selectively enriched DNA are determined by performing high-throughput sequencing.

The genetic data of the target individual and/or of the related individual can be transformed from a molecular state to an electronic state by measuring the appropriate genetic material using tools and or techniques taken from a group including, but not limited to: genotyping microarrays, and high throughput sequencing. Some high throughput sequencing methods include Sanger DNA sequencing, pyrosequencing, the ILLUMINA SOLEXA platform, ILLUMINA's GENOME ANALYZER, or APPLIED BIOSYSTEM's 454 sequencing platform, HELICOS's TRUE SINGLE MOLECULE SEQUENCING platform, HALCYON MOLECULAR's electron microscope sequencing method, or any other sequencing method. In some embodiments, the high throughput sequencing is performed on Illumina NextSeq, followed by demultiplexing and mapping to the human reference genome. All of these methods physically transform the genetic data stored in a sample of DNA into a set of genetic data that is typically stored in a memory device en route to being processed.

In some embodiments, the sequences of the selectively enriched DNA are determined by performing microarray analysis. In an embodiment, the microarray may be an ILLUMINA SNP microarray, or an AFFYMETRIX SNP microarray.

In some embodiments, the sequences of the selectively enriched DNA are determined by performing quantitative PCR (qPCR) or digital droplet PCR (ddPCR) analysis. qPCR measures the intensity of fluorescence at specific times (generally after every amplification cycle) to determine the relative amount of target molecule (DNA). ddPCR measures the actual number of molecules (target DNA) as each molecule is in one droplet, thus making it a discrete “digital” measurement. It provides absolute quantification because ddPCR measures the positive fraction of samples, which is the number of droplets that are fluorescing due to proper amplification. This positive fraction accurately indicates the initial amount of template nucleic acid.

### Non Invasive Prenatal Testing (NIPT)

Non-invasive prenatal tests (NIPT's) which utilize cfDNA from the plasma of pregnant women to detect chromosomal aneuploidies and microdeletions that may affect child health, are preferred embodiments of the methods described herein.

The present disclosure provides improvement to methods for determining the ploidy status of a chromosome in a gestating fetus from genotypic data measured from a mixed sample of DNA (i.e., DNA from the mother of the fetus, and DNA from the fetus) and optionally from genotypic data measured from a sample of genetic material from the mother and possibly also from the father. In some embodiments, the present disclosure provides methods for non-invasive prenatal testing (NIPT), specifically, determining the aneuploidy status of a fetus by observing allele measurements at a plurality of polymorphic loci in genotypic data measured on DNA mixtures, where certain allele measurements are indicative of an aneuploid fetus, while other allele measurements are indicative of a euploid fetus. Methods for determining ploidy status is described in detail in U.S. Patent Publications 20170242960 and 20180025109, and U.S. Pat. No. 9,163,282, incorporated herein.

In one aspect, the present disclosure relates to a method for non-invasive prenatal testing, comprising (a) isolating cfDNA from a biological sample of a pregnant woman, wherein the isolated cfDNA comprises a mixture of fetal cfDNA and maternal cfDNA; (b) optionally, ligating adaptors to the isolated cfDNA to obtain adaptor-ligated DNA, and/or amplifying the adaptor-ligated DNA to obtain amplified adaptor-ligated DNA; (c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of fetal cfDNA; (d) performing a multiplex amplification reaction to amplify at least 100 polymorphic loci on the selectively enriched DNA in one reaction mixture; and (e) determining the sequences of the selectively enriched DNA. In some embodiments, step (c) further comprises performing hybrid capture to select a plurality of polymorphic loci on the isolated cfDNA, the adaptor-ligated DNA, and/or amplified adaptor-ligated DNA prior to selectively enriching trinucleosomal, dinucleosomal, mononucleosomal or sub-mononucleosomal DNA. In some embodiments, step (c) comprises selectively enriching dinucleosomal, mononucleosomal or sub-mononucleosomal DNA from the isolated cfDNA, the adaptor-ligated DNA or the amplified adaptor-ligated DNA to obtain selectively enriched DNA. In some embodiments, step (c) comprises selectively enriching mononucleosomal or sub-mononucleosomal DNA from the isolated cfDNA, the adaptor-ligated DNA or the amplified adaptor-ligated DNA to obtain selectively enriched DNA. In some embodiments, wherein step (c) comprises selectively enriching sub-mononucleosomal DNA from the isolated cfDNA, the adaptor-ligated DNA or the amplified adaptor-ligated DNA to obtain selectively enriched DNA.

In some embodiments, the method comprises: a) extracting cfDNA from the maternal blood sample, wherein the DNA comprises cell-free DNA from the pregnant mother and from the fetus, wherein the target loci comprise more than 100, 200, 500, 1,000, 2,000, 5,000, or 10,000 polymorphic and/or non-polymorphic loci; (b) optionally, ligating adaptors to the isolated cfDNA to obtain adaptor-ligated DNA, and/or amplifying the adaptor-ligated DNA to obtain amplified adaptor-ligated DNA; (c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of fetal cfDNA; and d) enriching the cfDNA at the target loci by: i) for each of the target loci, hybridizing an upstream and a downstream ligation-mediated PCR probe to one strand of the cfDNA within a region of DNA that comprises the target locus; ii) ligating the upstream and the downstream ligation-mediated PCR probe that are hybridized to the same region of DNA comprising a target locus; and iii) amplifying ligated ligation-mediated PCR probes using PCR, thereby amplifying the target loci of the fetus, wherein the more than 100, 200, 500, 1,000, 2,000, 5,000, or 10,000 polymorphic and/or non-polymorphic loci are amplified in a single reaction mixture.

In some embodiments, the disclosure provides improved methods to perform prenatal evaluation of risks of aneuploidy by biochemical processing and digital analysis as described in Sparks et al., 18 Am J Obstet Gynecol 206:319.e1-9 (2012), incorporated herein. In some embodiments, the disclosed method first provides that the cfDNA fragments are labeled with biotin and bound to streptavidin-coated magnetic beads. Then, locus specific oligos are annealed to cfDNA. When the oligos hybridize to their cognate locus sequences in cfDNA, their termini form 2 nicks. Ligation of these nicks results in creation of ligation products capable of supporting amplification using universal polymerase chain reaction (UPCR) primers. Elution of this ligation product followed by UPCR with UPCR primers containing sample tags enables pooling and simultaneous sequencing of different UPCR products on a single lane. The UPCR primers may also contain universal tail sequences that support sequencing of locus-specific and sample-specific bases. In some embodiments, the UPCR primers contain universal tail sequences that support HiSeq (Illumina, San Diego, Calif.) cluster amplification.

In some embodiments, the sequence counts of the UPCR products may be normalized by systematically removing sample and assay biases, followed by analysis of polymorphic loci for fetal fraction as described in Sparks et al., 18 Am J Obstet Gynecol 206:319.e1-9 (2012). In some embodiments, the aneuploidy risk is estimated by using the FORTE algorithm as described in Sparks et al., 18 Am J Obstet Gynecol 206:319.e1-9 (2012).

In some embodiments, the method comprises: a) obtaining fetal and maternal chromosome segments from cfDNA in a maternal blood sample comprising chromosome segments from the one or more chromosomes of interest and chromosome segments from one or more reference chromosomes; (b) ligating adaptors to the isolated cfDNA to obtain adaptor-ligated DNA, and optionally amplifying the adaptor-ligated DNA to obtain amplified adaptor-ligated DNA; (c) selectively enriching trinucleosomal, dinucleosomal, mononucleosomal or sub-mononucleosomal DNA from the adaptor-ligated DNA or the amplified adaptor-ligated DNA to obtain selectively enriched DNA, wherein the selectively enriched DNA comprises an increased fraction of fetal cfDNA; and d) measuring the amounts of chromosome segments from the one or more chromosomes of interest by massively-parallel sequencing or shotgun sequencing.

In some embodiments, the fraction of fetal cfDNA is increased by at least 10% in the selectively enriched DNA compared to the isolated cfDNA. In some embodiments, the fraction of fetal cfDNA is increased by at least 20%, at least 30%, at least 40%, at least 50%, at least 100%, at least 200%, or at least 300% in the selectively enriched DNA compared to the isolated cfDNA.

In some embodiments, the present disclosure provides a method for non-invasive prenatal testing, further comprising determining the presence of at least one fetal chromosomal abnormality based on the sequences of the selectively enriched DNA. In some embodiments, the fetal chromosomal abnormality comprises single nucleotide variant (SNV), copy number variation (CNV), single nucleotide polymorphism (SNP), and/or chromosomal rearrangement. In some embodiments, the chromosomal abnormality comprises trisomy of one or more chromosomes included in the test. In some embodiments, the chromosomal abnormality comprises trisomy at chromosome 13, 18, 21, X or Y.

In some embodiments, the present disclosure provides a method for non-invasive prenatal testing, wherein the biological sample is a blood, plasma, serum, or urine sample.

In some embodiments, the present disclosure provides a method for non-invasive prenatal testing, wherein step (b) comprises ligating adaptors to the isolated cfDNA to obtain adaptor-ligated DNA, and wherein step (c) comprises selectively enriching trinucleosomal, dinucleosomal, mononucleosomal or sub-mononucleosomal DNA from the adaptor-ligated DNA. In some embodiments, wherein step (b) comprises ligating adaptors to the isolated cfDNA to obtain adaptor-ligated DNA and amplifying the adaptor-ligated DNA to obtain amplified adaptor-ligated DNA, and wherein step (c) comprises selectively enriching trinucleosomal, dinucleosomal, mononucleosomal or sub-mononucleosomal DNA from the amplified adaptor-ligated DNA.

As used herein, the term ‘adaptors,’ or ‘ligation adaptors’ or ‘library tags’ are DNA molecules containing a universal priming sequence that can be covalently linked to the 5-prime and 3-prime end of a population of target double stranded DNA molecules. In some embodiments, the addition of the adapters provides universal priming sequences to the 5-prime and 3-prime end of the target population from which PCR amplification can take place, amplifying all molecules from the target population, using a single pair of amplification primers. Disclosed herein are methods that permit the targeted amplification of over a hundred to tens of thousands of target sequences (e.g. SNP loci) from genomic DNA obtained from plasma. The amplified sample may be relatively free of primer dimer products and have low allelic bias at target loci. If during or after amplification the products are appended with sequencing compatible adaptors, analysis of these products can be performed by sequencing. These methods are more fully described in U.S. Patent Publications 20170242960 and 20180025109, and U.S. Pat. No. 9,163,282, incorporated herein.

In some embodiments, the present disclosure provides a method for non-invasive prenatal testing, step (d) comprises amplifying at least 1000 polymorphic loci on the selectively enriched DNA in one reaction mixture. In some embodiments, step (d) comprises amplifying at least 2000 polymorphic loci on the selectively enriched DNA in one reaction mixture. In some embodiments, step (d) comprises amplifying at least 5000 polymorphic loci on the selectively enriched DNA in one reaction mixture. In some embodiments, step (d) comprises amplifying at least 10000 polymorphic loci on the selectively enriched DNA in one reaction mixture. In some embodiments, step (d) comprises amplifying at least 25000 polymorphic loci on the selectively enriched DNA in one reaction mixture. In some embodiments, step (d) comprises amplifying at least 50000 polymorphic loci on the selectively enriched DNA in one reaction mixture. In some embodiments, step (d) comprises amplifying at least 100000 polymorphic loci on the selectively enriched DNA in one reaction mixture. In some embodiments, step (d) comprises amplifying at least 150000 polymorphic loci on the selectively enriched DNA in one reaction mixture. In some embodiments, step (d) comprises amplifying at least 200000 polymorphic loci on the selectively enriched DNA in one reaction mixture.

### Methods for Monitoring Transplant Rejection

The present disclosure provides improvements to methods of quantifying the amount of donor-derived cell-free DNA (dd-cfDNA) in a blood sample of a transplant recipient

In one aspect, the present disclosure relates to a method for monitoring transplant rejection, comprising (a) isolating cfDNA from a biological sample of a transplant recipient, wherein the isolated cfDNA comprises a mixture of donor-derived cfDNA and recipient cfDNA; (b) optionally, ligating adaptors to the isolated cfDNA to obtain adaptor-ligated DNA, and/or amplifying the adaptor-ligated DNA to obtain amplified adaptor-ligated DNA; (c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of donor-derived cfDNA; (d) performing a multiplex amplification reaction to amplify at least 100 polymorphic loci on the selectively enriched DNA in one reaction mixture; and (e) determining the sequences of the selectively enriched DNA.

In one embodiment, the fraction of donor-derived cfDNA is increased by at least 20% in the selectively enriched DNA compared to the isolated cfDNA. In one embodiment, the fraction of donor-derived cfDNA is increased by at least 30% in the selectively enriched DNA compared to the isolated cfDNA. In one embodiment, the fraction of donor-derived cfDNA is increased by at least 40% in the selectively enriched DNA compared to the isolated cfDNA. In one embodiment, the fraction of donor-derived cfDNA is increased by at least 50% in the selectively enriched DNA compared to the isolated cfDNA. In one embodiment, the fraction of donor-derived cfDNA is increased by at least 100% in the selectively enriched DNA compared to the isolated cfDNA. In one embodiment, the fraction of donor-derived cfDNA is increased by at least 200% in the selectively enriched DNA compared to the isolated cfDNA. In one embodiment, the fraction of donor-derived cfDNA is increased by at least 300% in the selectively enriched DNA compared to the isolated cfDNA. In one embodiment, the fraction of donor-derived cfDNA is increased by at least 400% in the selectively enriched DNA compared to the isolated cfDNA. In one embodiment, the fraction of donor-derived cfDNA is increased by at least 500% in the selectively enriched DNA compared to the isolated cfDNA.

In some embodiments, the method for monitoring transplant rejection further comprises quantifying the amount of donor-derived cfDNA. In one further embodiment, the present invention relates to a method of quantifying the amount of donor-derived cell-free DNA (dd-cfDNA) in a blood sample of a transplant recipient, comprising: extracting DNA from the blood sample of the transplant recipient, wherein the DNA comprises donor-derived cell-free DNA and recipient-derived cell-free DNA; performing targeted amplification at 500-50,000 target loci in a single reaction volume using 500-50,000 primer pairs, wherein the target loci comprise polymorphic loci and non-polymorphic loci, and wherein each primer pair is designed to amplify a target sequence of no more than 100 bp; and quantifying the amount of donor-derived cell-free DNA in the amplification products.

In some embodiments, the method for monitoring transplant rejection further comprises determining the likelihood of transplant rejection based on the amount of donor-derived cfDNA. In one embodiment, this disclosure relates to quantifying the amount of donor-derived cell-free DNA in the biological sample, wherein a greater amount of dd-cfDNA indicates a greater likelihood of transplant rejection. In some embodiments, the biological sample is a blood, plasma, serum, or urine sample.

In some embodiments, step (b) of the method for monitoring transplant rejection comprises ligating adaptors to the isolated cfDNA to obtain adaptor-ligated DNA, and step (c) comprises selectively enriching trinucleosomal, dinucleosomal, mononucleosomal or sub-mononucleosomal DNA from the adaptor-ligated DNA. In some embodiments, step (b) comprises ligating adaptors to the isolated cfDNA to obtain adaptor-ligated DNA and amplifying the adaptor-ligated DNA to obtain amplified adaptor-ligated DNA, and wherein step (c) comprises selectively enriching trinucleosomal, dinucleosomal, mononucleosomal or sub-mononucleosomal DNA from the amplified adaptor-ligated DNA. Methods of ligating adaptors to the isolated cfDNA fragments and methods of selectively enriching mononucleosomal or sub-mononucleosomal DNA from the adaptor-ligated DNA are described elsewhere herein.

Performing multiplex amplification as recited in step (d) of the method has been described elsewhere herein.

In some embodiments, step (e) of the method for monitoring transplant rejection comprises performing high-throughput sequencing, microarray, qPCR or ddPCR analysis as described elsewhere herein.

In some embodiments, step (c) further comprises performing hybrid capture to select a plurality of polymorphic loci on the isolated cfDNA, the adaptor-ligated DNA, and/or amplified adaptor-ligated DNA prior to selectively enriching trinucleosomal, dinucleosomal, mononucleosomal or sub-mononucleosomal DNA.

In some embodiments, step (c) comprises selectively enriching dinucleosomal, mononucleosomal or sub-mononucleosomal DNA from the isolated cfDNA, the adaptor-ligated DNA or the amplified adaptor-ligated DNA to obtain selectively enriched DNA. In some embodiments, step (c) comprises selectively enriching mononucleosomal or sub-mononucleosomal DNA from the isolated cfDNA, the adaptor-ligated DNA or the amplified adaptor-ligated DNA to obtain selectively enriched DNA. In some embodiments, wherein step (c) comprises selectively enriching sub-mononucleosomal DNA from the isolated cfDNA, the adaptor-ligated DNA or the amplified adaptor-ligated DNA to obtain selectively enriched DNA.

In some embodiments, the method for monitoring transplant rejection comprises longitudinally collecting one or more biological samples from the transplant recipient after transplantation, and repeating steps (a)-(e) for each biological samples longitudinally collected. The inclusion of longitudinal data enabled a unique evaluation of the natural variability of dd-cfDNA in transplant patients over time. In some embodiments, the method comprises longitudinally collecting a plurality of blood samples from the transplant recipient after transplantation, and repeating steps (a) to (e) for each biological sample collected. In some embodiments, the method comprises collecting and analyzing biological samples from the transplant recipient for a time period of about three months, or about six months, or about twelve months, or about eighteen months, or about twenty-four months, etc. In some embodiments, the method comprises collecting blood samples from the transplant recipient at an interval of about one week, or about two weeks, or about three weeks, or about one month, or about two months, or about three months, etc.

In some embodiments, the method disclosed herein is able to detect the presence or absence of biological phenomenon or medical condition using a maximum likelihood method or the closely related maximum a posteriori (MAP) technique. In an embodiment, a method is disclosed for determining the transplant status in a transplant recipient that involves taking any method currently known in the art that uses a single hypothesis rejection technique and reformulating it such that it uses a MLE or MAP technique. Informatics methods useful and relevant to the methods disclosed herein can be found in U.S. Patent Publication No. 20180025109, incorporated by reference herein, wherein the informatics methods are disclosed in the context of determination of genetic state of a fetus via non-invasive prenatal testing.

Additional disclosure regarding methods for monitoring transplant rejection are provided in U.S. Prov. App. 62/693,833 filed Jul. 3, 2018, U.S. Prov. App. 62/715,178 filed Aug. 6, 2018, and U.S. Prov. App. 62/781,882 filed Dec. 19, 2018, which are incorporated herein by reference in their entirety.

### Methods of Monitoring Relapse or Metastasis of Cancer

In one aspect, this disclosure relates to improved methods for monitoring relapse or metastasis of cancer by including a step selectively enriching trinucleosomal, dinucleosomal, mononucleosomal or sub-mononucleosomal DNA from the isolated cfDNA.

In one embodiments, this disclosure provides a method for monitoring relapse or metastasis of cancer, comprising (a) isolating cfDNA from a biological sample of a subject diagnosed with cancer; (b) optionally, ligating adaptors to the isolated cfDNA to obtain adaptor-ligated DNA, and/or amplifying the adaptor-ligated DNA to obtain amplified adaptor-ligated DNA; (c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of circulating tumor DNA (ctDNA); (d) performing a multiplex amplification reaction to amplify a plurality of patient-specific somatic mutations on the selectively enriched DNA in one reaction mixture, wherein the patient-specific somatic mutations are identified in a tumor sample of the subject; and (e) determining the sequences of the selectively enriched DNA.

In some embodiments, step (c) further comprises performing hybrid capture to select a plurality of polymorphic loci on the isolated cfDNA, the adaptor-ligated DNA, and/or amplified adaptor-ligated DNA prior to selectively enriching trinucleosomal, dinucleosomal, mononucleosomal or sub-mononucleosomal DNA.

In some embodiments, step (c) comprises selectively enriching dinucleosomal, mononucleosomal or sub-mononucleosomal DNA from the isolated cfDNA, the adaptor-ligated DNA or the amplified adaptor-ligated DNA to obtain selectively enriched DNA. In some embodiments, step (c) comprises selectively enriching mononucleosomal or sub-mononucleosomal DNA from the isolated cfDNA, the adaptor-ligated DNA or the amplified adaptor-ligated DNA to obtain selectively enriched DNA. In some embodiments, wherein step (c) comprises selectively enriching sub-mononucleosomal DNA from the isolated cfDNA, the adaptor-ligated DNA or the amplified adaptor-ligated DNA to obtain selectively enriched DNA.

In some embodiments, the fraction of fetal cfDNA is increased by at least 20% in the selectively enriched DNA compared to the isolated cfDNA. In some embodiments, the fraction of fetal cfDNA is increased by at least 30% in the selectively enriched DNA compared to the isolated cfDNA. In some embodiments, the fraction of fetal cfDNA is increased by at least 40% in the selectively enriched DNA compared to the isolated cfDNA. In some embodiments, the fraction of fetal cfDNA is increased by at least 50% in the selectively enriched DNA compared to the isolated cfDNA. In some embodiments, the fraction of fetal cfDNA is increased by at least 100% in the selectively enriched DNA compared to the isolated cfDNA. In some embodiments, the fraction of fetal cfDNA is increased by at least 200% in the selectively enriched DNA compared to the isolated cfDNA. In some embodiments, the fraction of fetal cfDNA is increased by at least 300% in the selectively enriched DNA compared to the isolated cfDNA. In some embodiments, the fraction of fetal cfDNA is increased by at least 400% in the selectively enriched DNA compared to the isolated cfDNA. In some embodiments, the fraction of fetal cfDNA is increased by at least 500% in the selectively enriched DNA compared to the isolated cfDNA.

Accordingly, provided herein in one embodiment, is a method for determining the single nucleotide variants present in a cancer (e.g., breast cancer, bladder cancer, or colorectal cancer) by determining the patient-specific somatic mutations present in a ctDNA sample from an individual, such as an individual having or suspected of having cancer (e.g., breast cancer, bladder cancer, or colorectal cancer).

The terms “cancer” and “cancerous” refer to or describe the physiological condition in animals that is typically characterized by unregulated cell growth. A “tumor” comprises one or more cancerous cells. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of the method for monitoring relapse or metastasis of cancer, the detection of two or more patient-specific somatic mutations in the selectively enriched DNA is indicative of relapse or metastasis of cancer. In some embodiments, the patient-specific somatic mutations comprise single nucleotide variant (SNV), copy number variation (CNV), and/or chromosomal rearrangement. The presence of 1, 2, 3, 4, 5, 6, 7, 8, 9, 10, 11, 12, 13, 14, or 15 SNVs on the low end of the range, and 2, 3, 4, 5, 6, 7, 8, 9, 10, 11, 12, 13, 14, 15, 16, 17, 18, 19, 20, 21, 22, 23, 24, 25, 30, 40, or 50 SNVs on the high end of the range, in the sample at the plurality of single nucleotide loci is indicative of the presence of cancer (e.g., breast cancer, bladder cancer, or colorectal cancer). In some embodiments, at least 2 or at least 5 SNVs are detected and the presence of the at least 2 or at least 5 SNVs is indicative of early relapse or metastasis of breast cancer, bladder cancer, or colorectal cancer. In some embodiments, the SNVs are single nucleotide polymorphisms (SNPs).

In some embodiments of the method for monitoring relapse or metastasis of cancer, the biological sample is a blood, plasma, serum, or urine sample.

In some embodiments of the method for monitoring relapse or metastasis of cancer, step (b) comprises ligating adaptors to the isolated cfDNA to obtain adaptor-ligated DNA, and step (c) comprises selectively enriching trinucleosomal, dinucleosomal, mononucleosomal or sub-mononucleosomal DNA from the adaptor-ligated DNA. In some embodiments, step (b) comprises ligating adaptors to the isolated cfDNA to obtain adaptor-ligated DNA and amplifying the adaptor-ligated DNA to obtain amplified adaptor-ligated DNA, and step (c) comprises selectively enriching trinucleosomal, dinucleosomal, mononucleosomal or sub-mononucleosomal DNA from the amplified adaptor-ligated DNA. Methods of ligating adaptors to DNA fragments and selectively enriching trinucleosomal, dinucleosomal, mononucleosomal or sub-mononucleosomal DNA from the adaptor-ligated DNA are described elsewhere herein.

In some embodiments of the method for monitoring relapse or metastasis of cancer, step (c) comprises performing size selection by gel electrophoresis, paramagnetic beads, spin column, salt precipitation, or biased amplification. The methods of size selection are described elsewhere herein.

In some embodiments of the method for monitoring relapse or metastasis of cancer, step (e) comprises performing high-throughput sequencing, microarray, qPCR or ddPCR analysis as described elsewhere herein.

In some embodiments of the method for monitoring relapse or metastasis of cancer, the method comprises longitudinally collecting one or more biological samples from the subject after the patient has been treated with surgery, first-line chemotherapy, and/or adjuvant therapy, and repeating steps (a)-(e) for each biological samples longitudinally collected. Accordingly, in some embodiments, the method comprising collecting and sequencing blood or urine samples from the patient longitudinally.

In some embodiments, the present disclosure relates to longitudinally collecting one or more blood or urine samples from the patient after the patient has been treated with surgery, first-line chemotherapy, and/or adjuvant therapy; generating a set of amplicons by performing a multiplex amplification reaction on nucleic acids isolated from each blood or urine sample or a fraction thereof, wherein each amplicon of the set of amplicons spans at least one single nucleotide variant locus of the set of patient-specific single nucleotide variant loci associated with the breast cancer, bladder cancer, or colorectal cancer; and determining the sequence of at least a segment of each amplicon of the set of amplicons that comprises a patient-specific single nucleotide variant locus, wherein detection of one or more (or two or more, or three or more, or four or more, or five or more, or six or more, or seven or more, or eight or more, or nine or more, or ten or more) patient-specific single nucleotide variants from the blood or urine sample is indicative of early relapse or metastasis of breast cancer, bladder cancer, or colorectal cancer.

Additional disclosure regarding methods for monitoring cancer relapse or metastasis are provided in U.S. Prov. App. 62/657,727 filed Apr. 14, 2018, U.S. Prov. App. 62/669,330 filed May 9, 2018, U.S. Prov. App. 62/693,843 filed Jul. 3, 2018, U.S. Prov. App. 62/715,143 filed Aug. 6, 2018, U.S. Prov. App. 62/746,210 filed Oct. 16, 2018, and U.S. Prov. App. 62/777,973 filed Dec. 11, 2018, which are incorporated herein by reference in their entirety.

### Molecular Barcodes

In some embodiments, the adaptors or primers describe herein may comprise one or more molecular barcodes. Molecular barcodes or molecular indexing sequences have been used in next generation sequencing to reduce quantitative bias introduced by replication, by tagging each nucleic acid fragment with a molecular barcode or molecular indexing sequence. Sequence reads that have different molecular barcodes or molecular indexing sequences represent different original nucleic acid molecules. By referencing the molecular barcodes or molecular indexing sequences, PCR artifacts, such as sequence changes generated by polymerase errors that are not present in the original nucleic acid molecules can be identified and separated from real variants/mutations present in the original nucleic acid molecules.

In some embodiments, molecular barcodes are introduced by ligating adaptors carrying the molecular barcodes to the isolated cfDNA to obtain adaptor-ligated and molecular barcoded DNA. In some embodiments, molecular barcodes are introduced by amplifying the adaptor-ligated DNA with primers carrying the molecular barcodes to obtain amplified adaptor-ligated and molecular barcoded DNA.

In some embodiments, the molecular barcoding adaptor or primers may comprise a universal sequence, followed by a molecular barcode region, optionally followed by a target specific sequence in the case of a primer. The sequence 5′ of molecular barcode may be used for subsequence PCR amplification or sequencing and may comprise sequences useful in the conversion of the amplicon to a library for sequencing. The random molecular barcode sequence could be generated in a multitude of ways. The preferred method synthesizes the molecule tagging adaptor or primer in such a way as to include all four bases to the reaction during synthesis of the barcode region. All or various combinations of bases may be specified using the IUPAC DNA ambiguity codes. In this manner the synthesized collection of molecules will contain a random mixture of sequences in the molecular barcode region. The length of the barcode region will determine how many adaptors or primers will contain unique barcodes. The number of unique sequences is related to the length of the barcode region as N^{L} where N is the number of bases, typically 4, and L is the length of the barcode. A barcode of five bases can yield up to 1024 unique sequences; a barcode of eight bases can yield 65536 unique barcodes. In an embodiment, the DNA can be measured by a sequencing method, where the sequence data represents the sequence of a single molecule. This can include methods in which single molecules are sequenced directly or methods in which single molecules are amplified to form clones detectable by the sequence instrument, but that still represent single molecules, herein called clonal sequencing.

In some embodiments, the molecular barcodes described herein are Molecular Index Tags (“MITs”), which are attached to a population of nucleic acid molecules from a sample to identify individual sample nucleic acid molecules from the population of nucleic acid molecules (i.e. members of the population) after sample processing for a sequencing reaction. MITs are described in detail in U.S. Pat. No. 10,011,870 to Zimmermann et al., which is incorporated herein by reference in its entirety. Unlike prior art methods that relate to unique identifiers and teach having a diversity of unique identifiers that is greater than the number of sample nucleic acid molecules in a sample in order to tag each sample nucleic acid molecule with a unique identifier, the present disclosure typically involves many more sample nucleic acid molecules than the diversity of MITs in a set of MITs. In fact, methods and compositions herein can include more than 1,000, 1×10⁶, 1×10⁹, or even more starting molecules for each different MIT in a set of MITs. Yet the methods can still identify individual sample nucleic acid molecules that give rise to a tagged nucleic acid molecule after amplification.

In the methods and compositions herein, the diversity of the set of MITs is advantageously less than the total number of sample nucleic acid molecules that span a target locus but the diversity of the possible combinations of attached MITs using the set of MITs is greater than the total number of sample nucleic acid molecules that span a target locus. Typically, to improve the identifying capability of the set of MITs, at least two MITs are attached to a sample nucleic acid molecule to form a tagged nucleic acid molecule. The sequences of attached MITs determined from sequencing reads can be used to identify clonally amplified identical copies of the same sample nucleic acid molecule that are attached to different solid supports or different regions of a solid support during sample preparation for the sequencing reaction. The sequences of tagged nucleic acid molecules can be compiled, compared, and used to differentiate nucleotide mutations incurred during amplification from nucleotide differences present in the initial sample nucleic acid molecules.

Sets of MITs in the present disclosure typically have a lower diversity than the total number of sample nucleic acid molecules, whereas many prior methods utilized sets of “unique identifiers” where the diversity of the unique identifiers was greater than the total number of sample nucleic acid molecules. Yet MITs of the present disclosure retain sufficient tracking power by including a diversity of possible combinations of attached MITs using the set of MITs that is greater than the total number of sample nucleic acid molecules that span a target locus. This lower diversity for a set of MITs of the present disclosure significantly reduces the cost and manufacturing complexity associated with generating and/or obtaining sets of tracking tags. Although the total number of MIT molecules in a reaction mixture is typically greater than the total number of sample nucleic acid molecules, the diversity of the set of MITs is far less than the total number of sample nucleic acid molecules, which substantially lowers the cost and simplifies the manufacturability over prior art methods. Thus, a set of MIT's can include a diversity of as few as 3, 4, 5, 10, 25, 50, or 100 different MITs on the low end of the range and 10, 25, 50, 100, 200, 250, 500, or 1000 MITs on the high end of the range, for example. Accordingly, in the present disclosure this relatively low diversity of MITs results in a far lower diversity of MITs than the total number of sample nucleic acid molecules, which in combination with a greater total number of MITs in the reaction mixture than total sample nucleic acid molecules and a higher diversity in the possible combinations of any 2 MITs of the set of MITs than the number of sample nucleic acid molecules that span a target locus, provides a particularly advantageous embodiment that is cost-effective and very effective with complex samples isolated from nature.

In some embodiments, the population of nucleic acid molecules has not been amplified in vitro before attaching the MITs and can include between 1×10⁸ and 1×10¹³, or in some embodiments, between 1×10⁹ and 1×10¹² or between 1×10¹⁰ and 1×10¹², sample nucleic acid molecules. In some embodiments, a reaction mixture is formed including the population of nucleic acid molecules and a set of MITs, wherein the total number of nucleic acid molecules in the population of nucleic acid molecules is greater than the diversity of MITs in the set of MITs and wherein there are at least three MITs in the set. In some embodiments, the diversity of the possible combinations of attached MITs using the set of MITs is more than the total number of sample nucleic acid molecules that span a target locus and less than the total number of sample nucleic acid molecules in the population. In some embodiments, the diversity of set of MITs can include between 10 and 500 MITs with different sequences. The ratio of the total number of nucleic acid molecules in the population of nucleic acid molecules in the sample to the diversity of MITs in the set, in certain methods and compositions herein, can be between 1,000:1 and 1,000,000,000:1. The ratio of the diversity of the possible combinations of attached MITs using the set of MITs to the total number of sample nucleic acid molecules that span a target locus can be between 1.01:1 and 10:1. The MITs typically are composed at least in part of an oligonucleotide between 4 and 20 nucleotides in length as discussed in more detail herein. The set of MITs can be designed such that the sequences of all the MITs in the set differ from each other by at least 2, 3, 4, or 5 nucleotides.

In some embodiments, provided herein, at least one (e.g. 2, 3, 5, 10, 20, 30, 50, 100) MIT from the set of MITs are attached to each nucleic acid molecule or to a segment of each nucleic acid molecule of the population of nucleic acid molecules to form a population of tagged nucleic acid molecules. MITs can be attached to a sample nucleic acid molecule in various configurations, as discussed further herein. For example, after attachment one MIT can be located on the 5′ terminus of the tagged nucleic acid molecules or 5′ to the sample nucleic acid segment of some, most, or typically each of the tagged nucleic acid molecules, and/or another MIT can be located 3′ to the sample nucleic acid segment of some, most, or typically each of the tagged nucleic acid molecules. In other embodiments, at least two MITs are located 5′ and/or 3′ to the sample nucleic acid segments of the tagged nucleic acid molecules, or 5′ and/or 3′ to the sample nucleic acid segment of some, most, or typically each of the tagged nucleic acid molecules. Two MITs can be added to either the 5′ or 3′ by including both on the same polynucleotide segment before attaching or by performing separate reactions. For example, PCR can be performed with primers that bind to specific sequences within the sample nucleic acid molecules and include a region 5′ to the sequence-specific region that encodes two MITs. In some embodiments, at least one copy of each MIT of the set of MITs is attached to a sample nucleic acid molecule, two copies of at least one MIT are each attached to a different sample nucleic acid molecule, and/or at least two sample nucleic acid molecules with the same or substantially the same sequence have at least one different MIT attached. A skilled artisan will identify methods for attaching MITs to nucleic acid molecules of a population of nucleic acid molecules. For example, MITs can be attached through ligation or appended 5′ to an internal sequence binding site of a PCR primer and attached during a PCR reaction as discussed in more detail herein.

After or while MITs are attached to sample nucleic acids to form tagged nucleic acid molecules, the population of tagged nucleic acid molecules are typically amplified to create a library of tagged nucleic acid molecules. Methods for amplification to generate a library, including those particularly relevant to a high-throughput sequencing workflow, are known in the art. For example, such amplification can be a PCR-based library preparation. These methods can further include clonally amplifying the library of tagged nucleic acid molecules onto one or more solid supports using PCR or another amplification method such as an isothermal method. Methods for generating clonally amplified libraries onto solid supports in high-throughput sequencing sample preparation workflows are known in the art. Additional amplification steps, such as a multiplex amplification reaction in which a subset of the population of sample nucleic acid molecules are amplified, can be included in methods for identifying sample nucleic acids provided herein as well.

In some embodiments, a nucleotide sequence of the MITs and at least a portion of the sample nucleic acid molecule segments of some, most, or all (e.g. at least 2, 3, 4, 5, 6, 7, 8, 9, 10, 20, 25, 50, 75, 100, 150, 200, 250, 500, 1,000, 2,500, 5,000, 10,000, 15,000, 20,000, 25,000, 50,000, 100,000, 1,000,000, 5,000,000, 10,000,000, 25,000,000, 50,000,000, 100,000,000, 250,000,000, 500,000,000, 1×10⁹, 1×10¹⁰, 1×10¹¹, 1×10¹², or 1×10¹³ tagged nucleic acid molecules or between 10, 20, 25, 30, 40, 50, 60, 70, 80, or 90% of the tagged nucleic acid molecules on the low end of the range and 20, 25, 30, 40, 50, 60, 70, 80, or 90, 95, 96, 97, 98, 99, and 100% on the high end of the range) of the tagged nucleic acid molecules in the library of tagged nucleic acid molecules is then determined. The sequence of a first MIT and optionally a second MIT or more MITs on clonally amplified copies of a tagged nucleic acid molecule can be used to identify the individual sample nucleic acid molecule that gave rise to the clonally amplified tagged nucleic acid molecule in the library.

In some embodiments, sequences determined from tagged nucleic acid molecules sharing the same first and optionally the same second MIT can be used to identify amplification errors by differentiating amplification errors from true sequence differences at target loci in the sample nucleic acid molecules. For example, in some embodiments, the set of MITs are double stranded MITs that, for example, can be a portion of a partially or fully double-stranded adapter, such as a Y-adapter. In these embodiments, for every starting molecule, a Y-adapter preparation generates 2 daughter molecule types, one in a + and one in a − orientation. A true mutation in a sample molecule should have both daughter molecules paired with the same 2 MITs in these embodiments where the MITs are a double stranded adapter, or a portion thereof. Additionally, when the sequences for the tagged nucleic acid molecules are determined and bucketed by the MITs on the sequences into MIT nucleic acid segment families, considering the MIT sequence and optionally its complement for double-stranded MITs, and optionally considering at least a portion of the nucleic acid segment, most, and typically at least 75% in double-stranded MIT embodiments, of the nucleic acid segments in an MIT nucleic acid segment family will include the mutation if the starting molecule that gave rise to the tagged nucleic acid molecules had the mutation. In the event of an amplification (e.g. PCR) error, the worst-case scenario is that the error occurs in cycle 1 of the 1^{st} PCR. In these embodiments, an amplification error will cause 25% of the final product to contain the error (plus any additional accumulated error, but this should be <<1%). Therefore, in some embodiments, if an MIT nucleic acid segment family contains at least 75% reads for a particular mutation or polymorphic allele, for example, it can be concluded that the mutation or polymorphic allele is truly present in the sample nucleic acid molecule that gave rise to the tagged nucleic acid molecule. The later an error occurs in a sample preparation process, the lower the proportion of sequence reads that include the error in a set of sequencing reads grouped (i.e. bucketed) by MITs into a paired MIT nucleic acid segment family. For example, an error in a library preparation amplification will result in a higher percentage of sequences with the error in a paired MIT nucleic acid segment family, than an error in a subsequent amplification step in the workflow, such as a targeted multiplex amplification. An error in the final clonal amplification in a sequencing workflow creates the lowest percentage of nucleic acid molecules in a paired MIT nucleic acid segment family that includes the error.

In some embodiments disclosed herein, the ratio of the total number of the sample nucleic acid molecules to the diversity of the MITs in the set of MITs or the diversity of the possible combinations of attached MITs using the set of MITs can be between 10:1, 20:1, 30:1, 40:1, 50:1, 60:1, 70:1, 80:1, 90:1, 100:1200:1,300:1,400:1,500:1,600:1,700:1,800:1,900:1, 1,000:1, 2,000:1, 3,000:1, 4,000:1, 5,000:1, 6,000:1, 7,000:1, 8,000:1, 9,000:1, 10,000:1, 15,000:1, 20,000:1, 25,000:1, 30,000:1, 40,000:1, 50,000:1, 60,000:1, 70,000:1, 80,000:1, 90,000:1, 100,000:1, 200,000:1, 300,000:1, 400,000:1, 500,000:1, 600,000:1, 700,000:1, 800,000:1, 900,000:1, and 1,000,000:1 on the low end of the range and 100:1 200:1, 300:1, 400:1, 500:1, 600:1, 700:1, 800:1, 900:1, 1,000:1, 2,000:1, 3,000:1, 4,000:1, 5,000:1, 6,000:1, 7,000:1, 8,000:1, 9,000:1, 10,000:1, 15,000:1, 20,000:1, 25,000:1, 30,000:1, 40,000:1, 50,000:1, 60,000:1, 70,000:1, 80,000:1, 90,000:1, 100,000:1, 200,000:1, 300,000:1, 400,000:1, 500,000:1, 600,000:1, 700,000:1, 800,000:1, 900,000:1, 1,000,000:1, 2,000,000:1, 3,000,000:1, 4,000,000:1, 5,000,000:1, 6,000,000:1, 7,000,000:1, 8,000,000:1, 9,000,000:1, 10,000,000:1, 50,000,000:1, 100,000,000:1, and 1,000,000,000:1 on the high end of the range.

In some embodiments, the sample is a human cfDNA sample. In such a method, as disclosed herein, the diversity is between about 20 million and about 3 billion. In these embodiments, the ratio of the total number of sample nucleic acid molecules to the diversity of the set of MITs can be between 100,000:1, 1×10⁶:1, 1×10⁷:1, 2×10⁷:1, and 2.5×10⁷:1 on the low end of the range and 2×10⁷:1, 2.5×10⁷:1, 5×10⁷:1, 1×10⁸:1, 2.5×10⁸:1, 5×10⁸:1, and 1×10⁹:1 on the high end of the range.

In some embodiments, the diversity of possible combinations of attached MITs using the set of MITs is preferably greater than the total number of sample nucleic acid molecules that span a target locus. For example, if there are 100 copies of the human genome that have all been fragmented into 200 bp fragments such that there are approximately 15,000,000 fragments for each genome, then it is preferable that the diversity of possible combinations of MITs be greater than 100 (number of copies of each target locus) but less than 1,500,000,000 (total number of nucleic acid molecules). For example, the diversity of possible combinations of MITs can be greater than 100 but much less than 1,500,000,000, such as 200, 300, 400, 500, 600, 700, 800, 900, or 1,000 possible combinations of attached MITs. While the diversity of MITs in the set of MITs is less than the total number of nucleic acid molecules, the total number of MITs in the reaction mixture is in excess of the total number of nucleic acid molecules or nucleic acid molecule segments in the reaction mixture. For example, if there are 1,500,000,000 total nucleic acid molecules or nucleic acid molecule segments, then there will be more than 1,500,000,000 total MIT molecules in the reaction mixture. In some embodiments, the ratio of the diversity of MITs in the set of MITs can be lower than the number of nucleic acid molecules in a sample that span a target locus while the diversity of the possible combinations of attached MITs using the set of MITs can be greater than the number of nucleic acid molecules in the sample that span a target locus. For example, the ratio of the number of nucleic acid molecules in a sample that span a target locus to the diversity of MITs in the set of MITs can be at least 10:1, 25:1, 50:1, 100:1, 125:1, 150:1, or 200:1 and the ratio of the diversity of the possible combinations of attached MITs using the set of MITs to the number of nucleic acid molecules in the sample that span a target locus can be at least 1.01:1, 1.1:1, 2:1, 3:1, 4:1, 5:1, 6:1, 7:1, 8:1, 9:1, 10:1, 20:1, 25:1, 50:1, 100:1, 250:1, 500:1, or 1,000:1.

Typically, the diversity of MITs in the set of MITs is less than the total number of sample nucleic acid molecules that span a target locus whereas the diversity of the possible combinations of attached MITs is greater than the total number of sample nucleic acid molecules that span a target locus. In embodiments where 2 MITs are attached to sample nucleic acid molecules, the diversity of MITs in the set of MITs is less than the total number of sample nucleic acid molecules that span a target locus but greater than the square root of the total number of sample nucleic acid molecules that span a target locus. In some embodiments, the diversity of MITs is less than the total number of sample nucleic acid molecules that span a target locus but 1, 2, 3, 4, or 5 more than the square root of the total number of sample nucleic acid molecules that span a target locus. Thus, although the diversity of MITs is less than the total number of sample nucleic acid molecules that span a target locus, the total number of combinations of any 2 MITs is greater than the total number of sample nucleic acid molecules that span a target locus. The diversity of MITs in the set is typically less than one half the number of sample nucleic acid molecules than span a target locus in samples with at least 100 copies of each target locus. In some embodiments, the diversity of MITs in the set can be at least 1, 2, 3, 4, or 5 more than the square root of the total number of sample nucleic acid molecules that span a target locus but less than ⅕, 1/10, 1/20, 1/50, or 1/100 the total number of sample nucleic acid molecules that span a target locus. For samples with between 2,000 and 1,000,000 sample nucleic acid molecules that span a target locus, the number of MITs in the set does not exceed 1,000. For example, in a sample with 10,000 copies of the genome in a genomic DNA sample such as a circulating cell-free DNA sample such that the sample has 10,000 sample nucleic acid molecules that span a target locus, the diversity of MITs can be between 101 and 1,000, or between 101 and 500, or between 101 and 250. In some embodiments, the diversity of MITs in the set of MITs can be between the square root of the total number of sample nucleic acid molecules that span a target locus and 1, 10, 25, 50, 100, 125, 150, 200, 250, 300, 400, 500, 600, 700, 800, 900, or 1,000 less than the total number of sample nucleic acid molecules that span a target locus. In some embodiments, the diversity of MITs in the set of MITs can be between 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, and 80% of the number of sample nucleic acid molecules that span a target locus on the low end of the range and 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, and 99% of the number of sample nucleic acid molecules that span a target locus on the high end of the range.

In some embodiments, the ratio of the total number of MITs in the reaction mixture to the total number of sample nucleic acid molecules in the reaction mixture can be between 1.01, 1.1:1, 2:1, 3:1, 4:1, 5:1, 6:1, 7:1, 8:1, 9:1, 10:1, 25:1 50:1, 100:1, 200:1, 300:1, 400:1, 500:1, 600:1, 700:1, 800:1, 900:1, 1,000:1, 2,000:1, 3,000:1, 4,000:1, 5,000:1, 6,000:1, 7,000:1, 8,000:1, 9,000:1, and 10,000:1 on the low end of the range and 25:1 50:1, 100:1, 200:1, 300:1, 400:1, 500:1, 600:1, 700:1, 800:1, 900:1, 1,000:1, 2,000:1, 3,000:1, 4,000:1, 5,000:1, 6,000:1, 7,000:1, 8,000:1, 9,000:1, 10,000:1, 15,000:1, 20,000:1, 25,000:1, 30,000:1, 40,000:1, and 50,000:1 on the high end of the range. In some embodiments, the total number of MITs in the reaction mixture is at least 50%, 60%, 70%, 80%, 90%, 95%, 96%, 97%, 98% 99%, or 99.9% of the total number of sample nucleic acid molecules in the reaction mixture. In other embodiments, the ratio of the total number of MITs in the reaction mixture to the total number of sample nucleic acid molecules in the reaction mixture can be at least enough MITs for each sample nucleic acid molecule to have the appropriate number of MITs attached, i.e. 2:1 for 2 MITs being attached, 3:1 for 3 MITs, 4:1 for 4 MITs, 5:1 for 5 MITs, 6:1 for 6 MITs, 7:1 for 7 MITs, 8:1 for 8 MITs, 9:1 for 0 MITs, and 10:1 for 10 MITs.

In some embodiments, the ratio of the total number of MITs with identical sequences in the reaction mixture to the total number of nucleic acid segments in the reaction mixture can be between 0.1:1, 0.2:1, 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.25:1, 2.5:1, 2.75:1, 3:1, 3.5:1, 4:1, 4.5:1, and 5:1 on the low end of the range and 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.25:1, 2.5:1, 2.75:1, 3:1, 3.5:1, 4:1, 4.5:1, 5:1, 6:1, 7:1, 8:1, 9:1, 10:1, 20:1, 30:1, 40:1, 50:1, 60:1, 70:1, 80:1, 90:1, and 100:1 on the high end of the range.

The set of MITs can include, for example, at least three MITs or between 10 and 500 MITs. As discussed herein in some embodiments, nucleic acid molecules from the sample are added directly to the attachment reaction mixture without amplification. These sample nucleic acid molecules can be purified from a source, such as a living cell or organism, as disclosed herein, and then MITs can be attached without amplifying the nucleic acid molecules. In some embodiments, the sample nucleic acid molecules or nucleic acid segments can be amplified before attaching MITs. As discussed herein, in some embodiments, the nucleic acid molecules from the sample can be fragmented to generate sample nucleic acid segments. In some embodiments, other oligonucleotide sequences can be attached (e.g. ligated) to the ends of the sample nucleic acid molecules before the MITs are attached.

In some embodiments disclosed herein the ratio of sample nucleic acid molecules, nucleic acid segments, or fragments that include a target locus to MITs in the reaction mixture can be between 1.01:1, 1.05, 1.1:1, 1.2:1 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 15:1, 20:1, 25:1, 30:1, 35:1, 40:1, 45:1, and 50:1 on the low end and 5:1, 6:1, 7:1, 8:1, 9:1, 10:1, 15:1, 20:1, 25:1, 30:1, 35:1, 40:1, 45:1, 50:1 60:1, 70:1, 80:1, 90:1, 100:1, 125:1, 150:1, 175:1, 200:1, 300:1, 400:1 and 500:1 on the high end. For example, in some embodiments, the ratio of sample nucleic acid molecules, nucleic acid segments, or fragments with a specific target locus to MITs in the reaction mixture is between 5:1, 6:1, 7:1, 8:1, 9:1, 10:1, 15:1, 20:1, 25:1, 30:1, 35:1, 40:1, 45:1, and 50:1 on the low end and 20:1, 25:1, 30:1, 35:1, 40:1, 45:1, 50:1, 60:1, 70:1, 80:1, 90:1, 100:1, and 200:1 on the high end. In some embodiments, the ratio of sample nucleic acid molecules or nucleic acid segments to MITs in the reaction mixture can be between 25:1, 30:1, 35:1, 40:1, 45:1, 50:1 on the low end and 50:1 60:1, 70:1, 80:1, 90:1, 100:1 on the high end. In some embodiments, the diversity of the possible combinations of attached MITs can be greater than the number of sample nucleic acid molecules, nucleic acid segments, or fragments that span a target locus. For example, in some embodiments, the ratio of the diversity of the possible combinations of attached MITs to the number of sample nucleic acid molecules, nucleic acid segments, or fragments that span a target locus can be at least 1.01, 1.1:1, 2:1, 3:1, 4:1, 5:1, 6:1, 7:1, 8:1, 9:1, 10:1, 20:1, 25:1, 50:1, 100:1, 250:1, 500:1, or 1,000:1.

Reaction mixtures for tagging nucleic acid molecules with MITs (i.e. attaching nucleic acid molecules to MITs), as provided herein, can include additional reagents in addition to a population of sample nucleic acid molecules and a set of MITs. For example, the reaction mixtures for tagging can include a ligase or polymerase with suitable buffers at an appropriate pH, adenosine triphosphate (ATP) for ATP-dependent ligases or nicotinamide adenine dinucleotide for NAD-dependent ligases, deoxynucleoside triphosphates (dNTPs) for polymerases, and optionally molecular crowding reagents such as polyethylene glycol. In certain embodiments the reaction mixture can include a population of sample nucleic acid molecules, a set of MITs, and a polymerase or ligase, wherein the ratio of the number of sample nucleic acid molecules, nucleic acid segments, or fragments with a specific target locus to the number of MITs in the reaction mixture can be any of the ratios disclosed herein, for example between 2:1 and 100:1, or between 10:1 and 100:1 or between 25:1 and 75:1, or is between 40:1 and 60:1, or between 45:1 and 55:1, or between 49:1 and 51:1.

In some embodiments disclosed herein the number of different MITs (i.e. diversity) in the set of MITs can be between 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 250, 300, 350, 400, 450, 500, 600, 700, 800, 900, 1,000, 1,500, 2,000, 2,500, and 3,000 MITs with different sequences on the low end and 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 250, 300, 350, 400, 450, 500, 600, 700, 800, 900, 1,000, 2,000, 3,000, 4,000, and 5,000 MITs with different sequences on the high end. For example, the diversity of different MITs in the set of MITs can be between 20, 25, 30, 35, 40, 45, 50, 60, 70, 80, 90, and 100 different MIT sequences on the low end and 50, 60, 70, 80, 90, 100, 125, 150, 175, 200, 250, and 300 different MIT sequences on the high end. In some embodiments, the diversity of different MITs in the set of MITs can be between 50, 60, 70, 80, 90, 100, 125, and 150 different MIT sequences on the low end and 100, 125, 150, 175, 200, and 250 different MIT sequences on the high end. In some embodiments, the diversity of different MITs in the set of MITs can be between 3 and 1,000, or 10 and 500, or 50 and 250 different MIT sequences. In some embodiments, the diversity of possible combinations of attached MITs using the set of MITs can be between 4, 5, 6, 7, 8, 9, 10, 15, 20, 25, 30, 40, 50, 75, 100, 150, 200, 250, 300, 400, 500, and 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 250,000, 500,000, 1,000,000, possible combinations of attached MITs on the low end of the range and 10, 15, 20, 25, 30, 40, 50, 75, 100, 150, 200, 250, 300, 400, 500, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 250,000, 500,000, 1,000,000, 2,000,000, 3,000,000, 4,000,000, 5,000,000, 6,000,000, 7,000,000, 8,000,000, 9,000,000, and 10,000,000 possible combinations of attached MITs on the high end of the range.

The MITs in the set of MITs are typically all the same length. For example, in some embodiments, the MITs can be any length between 2, 3, 4, 5, 6, 7, 8, 9, 10, 11, 12, 13, 14, 15, 16, 17, 18, 19, and 20 nucleotides on the low end and 4, 5, 6, 7, 8, 9, 10, 11, 12, 13, 14, 15, 16, 17, 18, 19, 20, 21, 22, 23, 24, 25, 26, 27, 28, 29, and 30 nucleotides on the high end. In certain embodiments, the MITs are any length between 3, 4, 5, 6, 7, or 8 nucleotides on the low end and 5, 6, 7, 8, 9, 10, or 11 nucleotides on the high end. In some embodiments, the lengths of the MITs can be any length between 4, 5, or 6, nucleotides on the low end and 5, 6, or 7 nucleotides on the high end. In some embodiments, the length of the MITs is 5, 6, or 7 nucleotides.

As will be understood, a set of MITs typically includes many identical copies of each MIT member of the set. In some embodiments, a set of MITs includes between 10, 20, 25, 30, 40, 50, 100, 500, 1,000, 10,000, 50,000, and 100,000 times more copies on the low end of the range, and 100, 500, 1,000, 10,000, 50,000, 100,000, 250,000, 500,000 and 1,000,000 more copies on the high end of the range, than the total number of sample nucleic acid molecules that span a target locus. For example, in a human circulating cell-free DNA sample isolated from plasma, there can be a quantity of DNA fragments that includes, for example, 1,000-100,000 circulating fragments that span any target locus of the genome. In certain embodiments, there are no more than 1/10, ¼, ½, or ¾ as many copies of any given MIT as total unique MITs in a set of MITs. Between members of the set, there can be 1, 2, 3, 4, 5, 6, 7, 8, 9, or 10 differences between any sequence and the rest of the sequences. In some embodiments, the sequence of each MIT in the set differs from all the other MITs by at least 1, 2, 3, 4, 5, 6, 7, 8, 9, or 10 nucleotides. To reduce the chance of misidentifying an MIT, the set of MITs can be designed using methods a skilled artisan will recognize, such as taking into consideration the Hamming distances between all the MITs in the set of MITs. The Hamming distance measures the minimum number of substitutions required to change one string, or nucleotide sequence, into another. Here, the Hamming distance measures the minimum number of amplification errors required to transform one MIT sequence in a set into another MIT sequence from the same set. In certain embodiments, different MITs of the set of MITs have a Hamming distance of less than 1, 2, 3, 4, 5, 6, 7, 8, 9, or 10 between each other.

In certain embodiments, a set of isolated MITs as provided herein is one embodiment of the present disclosure. The set of isolated MITs can be a set of single stranded, or partially, or fully double stranded nucleic acid molecules, wherein each MIT is a portion of, or the entire, nucleic acid molecule of the set. In certain examples, provided herein is a set of Y-adapter (i.e. partially double-stranded) nucleic acids that each include a different MIT. The set of Y-adapter nucleic acids can each be identical except for the MIT portion. Multiple copies of the same Y-adapter MIT can be included in the set. The set can have a number and diversity of nucleic acid molecules as disclosed herein for a set of MITs. As a non-limiting example, the set can include 2, 5, 10, or 100 copies of between 50 and 500 MIT-containing Y-adapters, with each MIT segment between 4 and 8 nucleic acids in length and each MIT segment differing from the other MIT segments by at least 2 nucleotides, but contain identical sequences other than the MIT sequence. Further details regarding Y-adapter portion of the set of Y-adapters is provided herein.

In other embodiments, a reaction mixture that includes a set of MITs and a population of sample nucleic acid molecules is one embodiment of the present disclosure. Furthermore, such a composition can be part of numerous methods and other compositions provided herein. For example, in further embodiments, a reaction mixture can include a polymerase or ligase, appropriate buffers, and supplemental components as discussed in more detail herein. For any of these embodiments, the set of MITs can include between 25, 50, 100, 200, 250, 300, 400, 500, or 1,000 MITs on the low end of the range, and 100, 200, 250, 300, 400, 500, 1,000, 1,500, 2,000, 2,500, 5,000, 10,000, or 25,000 MITs on the high end of the range. For example, in some embodiments, a reaction mixture includes a set of between 10 and 500 MITs.

Molecular Index Tags (MITs) as discussed in more detail herein can be attached to sample nucleic acid molecules in the reaction mixture using methods that a skilled artisan will recognize. In some embodiments, the MITs can be attached alone, or without any additional oligonucleotide sequences. In some embodiments, the MITs can be part of a larger oligonucleotide that can further include other nucleotide sequences as discussed in more detail herein. For example, the oligonucleotide can also include primers specific for nucleic acid segments or universal primer binding sites, adapters such as sequencing adapters such as Y-adapters, library tags, ligation adapter tags, and combinations thereof. A skilled artisan will recognize how to incorporate various tags into oligonucleotides to generate tagged nucleic acid molecules useful for sequencing, especially high-throughput sequencing. The MITs of the present disclosure are advantageous in that they are more readily used with additional sequences, such as Y-adapter and/or universal sequences because the diversity of nucleic acid molecules is less, and therefore they can be more easily combined with additional sequences on an adapter to yield a smaller, and therefore more cost effective set of MIT-containing adapters.

In some embodiments, the MITs are attached such that one MIT is 5′ to the sample nucleic acid segment and one MIT is 3′ to the sample nucleic acid segment in the tagged nucleic acid molecule. For example, in some embodiments, the MITs can be attached directly to the 5′ and 3′ ends of the sample nucleic acid molecules using ligation. In some embodiments disclosed herein, ligation typically involves forming a reaction mixture with appropriate buffers, ions, and a suitable pH in which the population of sample nucleic acid molecules, the set of MITs, adenosine triphosphate, and a ligase are combined. A skilled artisan will understand how to form the reaction mixture and the various ligases available for use. In some embodiments, the nucleic acid molecules can have 3′ adenosine overhangs and the MITs can be located on double-stranded oligonucleotides having 5′ thymidine overhangs, such as directly adjacent to a 5′ thymidine.

In further embodiments, MITs provided herein can be included as part of Y-adapters before they are ligated to sample nucleic acid molecules. Y-adapters are well-known in the art and are used, for example, to more effectively provide primer binding sequences to the two ends of the nucleic acid molecules before high-throughput sequencing. Y-adapters are formed by annealing a first oligonucleotide and a second oligonucleotide where a 5′ segment of the first oligonucleotide and a 3′ segment of the second oligonucleotide are complementary and wherein a 3′ segment of the first oligonucleotide and a 5′ segment of the second oligonucleotide are not complementary. In some embodiments, Y-adapters include a base-paired, double-stranded polynucleotide segment and an unpaired, single-stranded polynucleotide segment distal to the site of ligation. The double-stranded polynucleotide segment can be between 3, 4, 5, 6, 7, 8, 9, 10, 11, 12, 13, 14, 15, 16, 17, 18, 19, or 20 nucleotides in length on the low end of the range and 4, 5, 6, 7, 8, 9, 10, 11, 12, 13, 14, 15, 16, 17, 18, 19, 20, 21, 22, 23, 24, 25, 26, 27, 28, 29, and 30 nucleotides in length on the high end of the range. The single-stranded polynucleotide segments on the first and second oligonucleotides can be between 3, 4, 5, 6, 7, 8, 9, 10, 11, 12, 13, 14, 15, 16, 17, 18, 19, or 20 nucleotides in length on the low end of the range and 4, 5, 6, 7, 8, 9, 10, 11, 12, 13, 14, 15, 16, 17, 18, 19, 20, 21, 22, 23, 24, 25, 26, 27, 28, 29, and 30 nucleotides in length on the high end of the range. In these embodiments, MITs are typically double stranded sequences added to the ends of Y-adapters, which are ligated to sample nucleic acid segments to be sequenced. In some embodiments, the non-complementary segments of the first and second oligonucleotides can be different lengths.

In some embodiments, double-stranded MITs attached by ligation will have the same MIT on both strands of the sample nucleic acid molecule. In certain aspects the tagged nucleic acid molecules derived from these two strands will be identified and used to generate paired MIT families. In downstream sequencing reactions, where single stranded nucleic acids are typically sequenced, an MIT family can be identified by identifying tagged nucleic acid molecules with identical or complementary MIT sequences. In these embodiments, the paired MIT families can be used to verify the presence of sequence differences in the initial sample nucleic acid molecule as discussed herein.

In some embodiments, MITs can be attached to the sample nucleic acid segment by being incorporated 5′ to forward and/or reverse PCR primers that bind sequences in the sample nucleic acid segment. In some embodiments, the MITs can be incorporated into universal forward and/or reverse PCR primers that bind universal primer binding sequences previously attached to the sample nucleic acid molecules. In some embodiments, the MITs can be attached using a combination of a universal forward or reverse primer with a 5′ MIT sequence and a forward or reverse PCR primer that bind internal binding sequences in the sample nucleic acid segment with a 5′ MIT sequence. After 2 cycles of PCR, sample nucleic acid molecules that have been amplified using both the forward and reverse primers with incorporated MIT sequences will have MITs attached 5′ to the sample nucleic acid segments and 3′ to the sample nucleic acid segments in each of the tagged nucleic acid molecules. In some embodiments, the PCR is done for 2, 3, 4, 5, 6, 7, 8, 9, or 10 cycles in the attachment step.

In some embodiments disclosed herein the two MITs on each tagged nucleic acid molecule can be attached using similar techniques such that both MITs are 5′ to the sample nucleic acid segments or both MITs are 3′ to the sample nucleic acid segments. For example, two MITs can be incorporated into the same oligonucleotide and ligated on one end of the sample nucleic acid molecule or two MITs can be present on the forward or reverse primer and the paired reverse or forward primer can have zero MITs. In other embodiments, more than two MITs can be attached with any combination of MITs attached to the 5′ and/or 3′ locations relative to the nucleic acid segments.

As discussed herein, other sequences can be attached to the sample nucleic acid molecules before, after, during, or with the MITs. For example, ligation adapters, often referred to as library tags or ligation adaptor tags (LTs), appended, with or without a universal primer binding sequence to be used in a subsequent universal amplification step. In some embodiments, the length of the oligonucleotide containing the MITs and other sequences can be between 5, 6, 7, 8, 9, 10, 11, 12, 13, 14, 15, 16, 17, 18, 29, 20, 21, 22, 23, 24, 25, 26, 27, 28, 29, 30, 35, 40, 45, 50, 55, 60, 65, 70, 75, 80, 85, 90, 95, and 100 nucleotides on the low end of the range and 10, 11, 12, 13, 14, 15, 16, 17, 18, 29, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, and 200 nucleotides on the high end of the range. In certain aspects the number of nucleotides in the MIT sequences can be a percentage of the number of nucleotides in the total sequence of the oligonucleotides that include MITs. For example, in some embodiments, the MIT can be at most 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of the total nucleotides of an oligonucleotide that is ligated to a sample nucleic acid molecule.

After attaching MITs to the sample nucleic acid molecules through a ligation or PCR reaction, it may be necessary to clean up the reaction mixture to remove undesirable components that could affect subsequent method steps. In some embodiments, the sample nucleic acid molecules can be purified away from the primers or ligases. In other embodiments, the proteins and primers can be digested with proteases and exonucleases using methods known in the art.

After attaching MITs to the sample nucleic acid molecules, a population of tagged nucleic acid molecules is generated, itself forming embodiments of the present disclosure. In some embodiments, the size ranges of the tagged nucleic acid molecules can be between 10, 20, 30, 40, 50, 60, 70, 80, 90, 100, 125, 150, 175, 200, 250, 300, 400, and 500 nucleotides on the low end of the range and 100, 125, 150, 175, 200, 250, 300, 400, 500, 600, 700, 800, 900, 1,000, 2,000, 3,000, 4,000, and 5,000 nucleotides on the high end of the range.

Such a population of tagged nucleic acid molecules can include between 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 225, 250, 300, 350, 400, 450, 500, 600, 700, 800, 900, 1,000, 2,000, 3,000, 4,000, 5,000, 6,000, 7,000, 8,000, 9,000, 10,000, 15,000, 20,000, 30,000, 40,000, 50,000, 100,000, 200,000, 300,000, 400,000, 500,000, 600,000, 700,000, 800,000, 900,000, 1,000,000, 1,250,000, 1,500,000, 2,000,000, 2,500,000, 3,000,000, 4,000,000, 5,000,000, 10,000,000, 20,000,000, 30,000,000, 40,000,000, 50,000,000, 50,000,000, 100,000,000, 200,000,000, 300,000,000, 400,000,000, 500,000,000, 600,000,000, 700,000,000, 800,000,000, 900,000,000, and 1,000,000,000 tagged nucleic acid molecules on the low end of the range and 10, 15, 20, 25, 30, 40, 50, 60, 70, 80, 90, 100, 150, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 40,000, 50,000, 100,000, 200,000, 300,000, 400,000, 500,000, 600,000, 700,000, 800,000, 900,000, 1,000,000, 1,250,000, 1,500,000, 2,000,000, 2,500,000, 3,000,000, 4,000,000, 5,000,000, 6,000,000, 7,000,000, 8,000,000, 9,000,000, 10,000,000, 20,000,000, 30,000,000, 40,000,000, 50,000,000, 100,000,000, 200,000,000, 300,000,000, 400,000,000, 500,000,000, 600,000,000, 700,000,000, 800,000,000, 900,000,000, 1,000,000,000, 2,000,000,000, 3,000,000,000, 4,000,000,000, 5,000,000,000, 6,000,000,000, 7,000,000,000, 8,000,000,000, 9,000,000,000, and 10,000,000,000, tagged nucleic acid molecules on the high end of the range. In some embodiments, the population of tagged nucleic acid molecules can include between 100,000,000, 200,000,000, 300,000,000, 400,000,000, 500,000,000, 600,000,000, 700,000,000, 800,000,000, 900,000,000, and 1,000,000,000 tagged nucleic acid molecules on the low end of the range and 500,000,000, 600,000,000, 700,000,000, 800,000,000, 900,000,000, 1,000,000,000, 2,000,000,000, 3,000,000,000, 4,000,000,000, 5,000,000,000 tagged nucleic acid molecules on the high end of the range.

In certain aspects a percentage of the total sample nucleic acid molecules in the population of sample nucleic acid molecules can be targeted to have MITs attached. In some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 99.9% of the sample nucleic acid molecules can be targeted to have MITs attached. In other aspects a percentage of the sample nucleic acid molecules in the population can have MITs successfully attached. In any of the embodiments disclosed herein at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 99.9% of the sample nucleic acid molecules can have MITs successfully attached to form the population of tagged nucleic acid molecules. In any of the embodiments disclosed herein at least 1, 2, 3, 4, 5, 6, 7, 8, 9, 10, 15, 20, 25, 30, 40, 50, 75, 100, 200, 300, 500, 600, 700, 800, 900, 1,000, 2,000, 3,000, 4,000, 5,000, 6,000, 7,000, 8,000, 9,000, 10,000, 15,000, 20,000, 30,000, 40,000, or 50,000 of the sample nucleic acid molecules can have MITs successfully attached to form the population of tagged nucleic acid molecules.

In some embodiments disclosed herein, MITs can be oligonucleotide sequences of ribonucleotides or deoxyribonucleotides linked through phosphodiester linkages. Nucleotides as disclosed herein can refer to both ribonucleotides and deoxyribonucleotides and a skilled artisan will recognize when either form is relevant for a particular application. In certain embodiments, the nucleotides can be selected from the group of naturally-occurring nucleotides consisting of adenosine, cytidine, guanosine, uridine, 5-methyluridine, deoxyadenosine, deoxycytidine, deoxyguanosine, deoxythymidine, and deoxyuridine. In some embodiments, the MITs can be non-natural nucleotides. Non-natural nucleotides can include: sets of nucleotides that bind to each other, such as, for example, d5SICS and dNaM; metal-coordinated bases such as, for example, 2,6-bis(ethylthiomethyl)pyridine (SPy) with a silver ion and mondentate pyridine (Py) with a copper ion; universal bases that can pair with more than one or any other base such as, for example, 2′-deoxyinosine derivatives, nitroazole analogues, and hydrophobic aromatic non-hydrogen-bonding bases; and xDNA nucleobases with expanded bases. In certain embodiments, the oligonucleotide sequences can be predetermined while in other embodiments, the oligonucleotide sequences can be degenerate.

In some embodiments, MITs include phosphodiester linkages between the natural sugars ribose and/or deoxyribose that are attached to the nucleobase. In some embodiments, non-natural linkages can be used. These linkages include, for example, phosphorothioate, boranophosphate, phosphonate, and triazole linkages. In some embodiments, combinations of the non-natural linkages and/or the phosphodiester linkages can be used. In some embodiments, peptide nucleic acids can be used wherein the sugar backbone is instead made of repeating N-(2-aminoethyl)-glycine units linked by peptide bonds. In any of the embodiments disclosed herein non-natural sugars can be used in place of the ribose or deoxyribose sugar. For example, threose can be used to generate α-(L)-threofuranosyl-(3′-2′) nucleic acids (TNA). Other linkage types and sugars will be apparent to a skilled artisan and can be used in any of the embodiments disclosed herein.

In some embodiments, nucleotides with extra bonds between atoms of the sugar can be used. For example, bridged or locked nucleic acids can be used in the MITs. These nucleic acids include a bond between the 2′-position and 4′-position of a ribose sugar.

In certain embodiments, the nucleotides incorporated into the sequence of the MIT can be appended with reactive linkers. At a later time, the reactive linkers can be mixed with an appropriately-tagged molecule in suitable conditions for the reaction to occur. For example, aminoallyl nucleotides can be appended that can react with molecules linked to a reactive leaving group such as succinimidyl ester and thiol-containing nucleotides can be appended that can react with molecules linked to a reactive leaving group such as maleimide. In other embodiments, biotin-linked nucleotides can be used in the sequence of the MIT that can bind streptavidin-tagged molecules.

Various combinations of the natural nucleotides, non-natural nucleotides, phosphodiester linkages, non-natural linkages, natural sugars, non-natural sugars, peptide nucleic acids, bridged nucleic acids, locked nucleic acids, and nucleotides with appended reactive linkers will be recognized by a skilled artisan and can be used to form MITs in any of the embodiments disclosed herein.

### WORKING EXAMPLES

### Example 1

This example showed that enriching the fetal fraction by size selecting for a subfraction of the mononucleosomal DNA peak resulted in a 2 to 5 fold fetal enrichment.

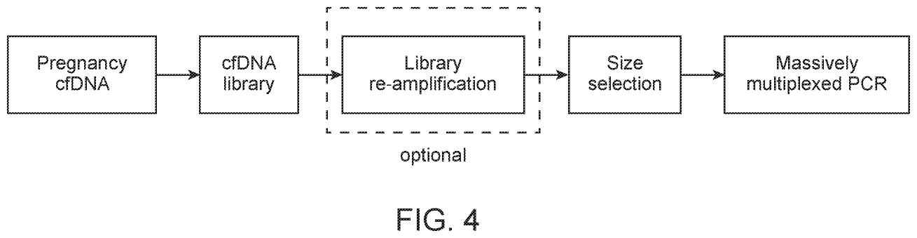

The overall workflow of this experiment is outlined in FIG. 4. Briefly, cell-free DNA (cfDNA) was isolated from 16 low risk samples and 4 samples with trisomy 21, which were estimated to have a low fetal fraction (most of them had less than 6% fetal fraction). Then end-repair, A-tailing, adaptor ligation, and PCR amplification were performed to create DNA libraries of each case. Size selection for mononucleosomal peak or subfraction of mononucleosomal peak was performed by using an automated gel electrophoresis system (Pippin™). A size selection of 100-237 basepairs (bp) range was applied to the 20 pregnancy libraries. The ligated adaptor had a size of 67 bp, so the size range of the cfDNA before ligation was therefore in the range from 33 to 170 bp. Alternatively, the size selection for mononucleosomal peak or subfraction of mononucleosomal peak can be performed without the library re-amplification PCR reaction (FIG. 4).

The recovered cfDNA library population for each case were processed through Natera's Panorama™ v3 pipeline and OneSTAR™. The cfDNA was preserved and analyzed in the single nucleotide polymorphism (SNP) based non-invasive prenatal test (NIPT) Panorama™ as described in Samango-Sprouse C, Banjevic M, Ryan A, et al. (2013) SNP-based non-invasive prenatal testing detects sex chromosome aneuploidies with high accuracy. Prenatal Diagnostics 33:643-9, and Hall M P, Hill M, Zimmermann, P B, et al (2014) Non-invasive prenatal detection of trisomy 13 using a single nucleotide polymorphism- and informatics-based approach. PLoS One 9:e96677, incorporated herein. The Panorama™ assay may be used to calculate the proportion of fetal to maternal SNP's, accurately reported as the percent child fraction estimate (% CFE).

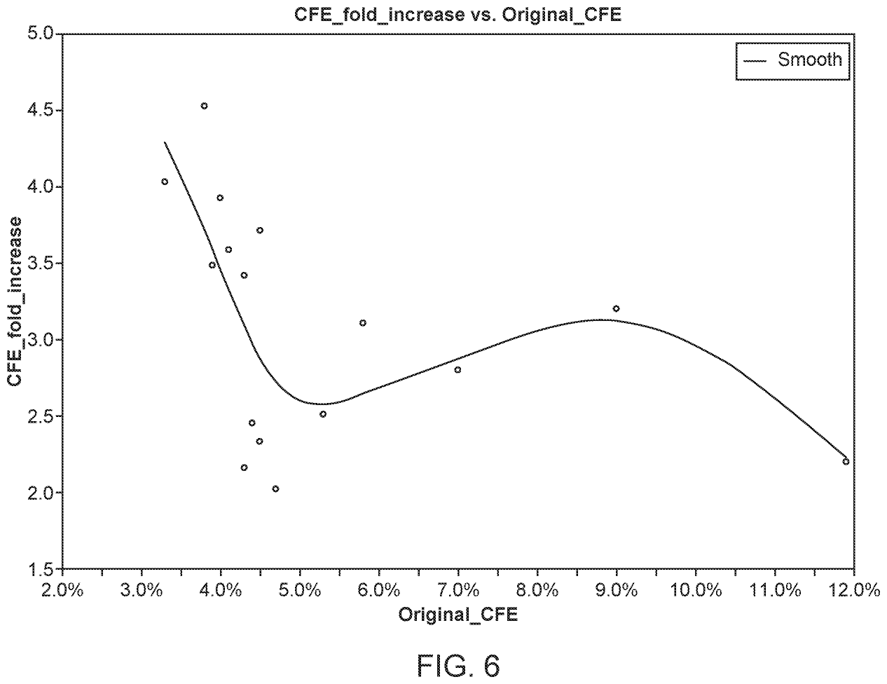

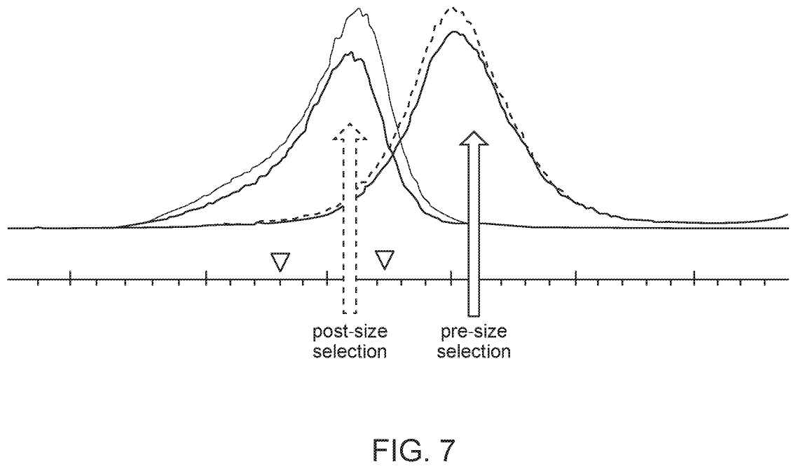

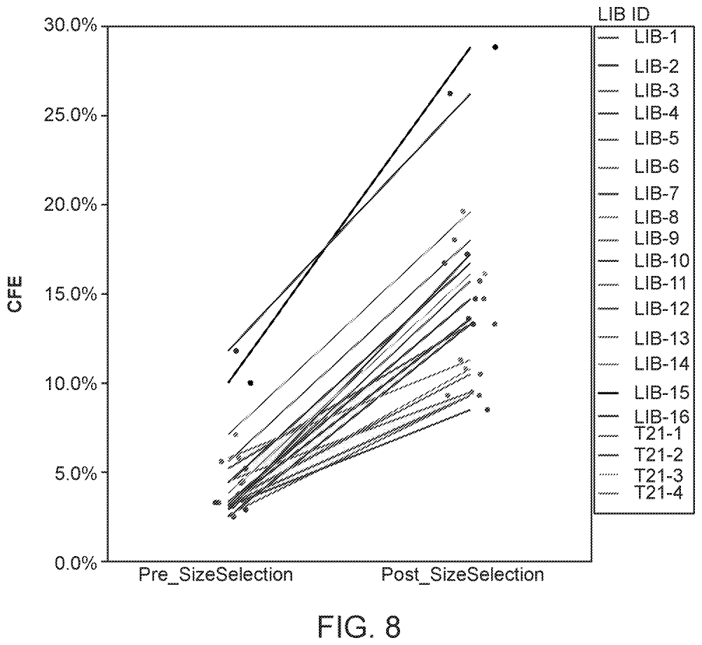

The determined % CFEs from the 20 samples are shown in FIG. 5 and FIG. 8. All samples showed a fetal enrichment of about 2 to 5 fold, and on average the size exclusion step resulted in an average fetal enrichment of about 3 fold. The enrichment for the fetal fraction was more pronounced in samples having low CFE in the original sample as shown in FIG. 6. The size distribution of 2 cfDNA samples pre-size selection (solid arrow on the right side) and post-size selection (dotted arrow on the left side) is shown in FIG. 7.

Determination of disomy/trisomy calling based on the post size selection samples were 100% confident and accurate. Statistical power is increased in post-size selection sample due to the child fraction increase.

1. A method for preparing a preparation of amplified DNA derived from a biological sample useful for determining the sequences of cell-free DNA (cfDNA), comprising
(a) isolating cfDNA from a biological sample of a subject;
(b) preparing a preparation of amplified DNA by: optionally, ligating adaptors to the isolated cfDNA to obtain adaptor-ligated DNA, and/or amplifying the adaptor-ligated DNA to obtain amplified adaptor-ligated DNA; and
selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA;
(c) analyzing the preparation of amplified DNA by determining the sequences of the selectively enriched DNA.

2. The method of claim 1, wherein the biological sample is a blood, plasma, serum, or urine sample.

3. The method of claim 1, wherein the preparing a preparation of amplified DNA comprises ligating adaptors to the isolated cfDNA to obtain adaptor-ligated DNA, and selectively enriching trinucleosomal, dinucleosomal, mononucleosomal or sub-mononucleosomal DNA from the adaptor-ligated DNA.

4. The method of claim 1, wherein the preparing a preparation of amplified DNA comprises ligating adaptors to the isolated cfDNA to obtain adaptor-ligated DNA and amplifying the adaptor-ligated DNA to obtain amplified adaptor-ligated DNA, and selectively enriching trinucleosomal, dinucleosomal, mononucleosomal or sub-mononucleosomal DNA from the amplified adaptor-ligated DNA.

5. The method of claim 1, wherein the selectively enriching comprises performing size selection by gel electrophoresis, paramagnetic beads, spin column, salt precipitation, or biased amplification.

6. The method of claim 1, wherein the preparing a preparation of amplified DNA further comprises performing a multiplex amplification reaction to amplify a plurality of polymorphic loci on the selectively enriched DNA in one reaction mixture.

7. The method of claim 1, wherein the preparing a preparation of amplified DNA further comprises performing hybrid capture to select a plurality of polymorphic loci on the selectively enriched DNA.

8. The method of claim 1, wherein the analyzing the preparation of amplified DNA comprises performing high-throughput sequencing, microarray analysis, or qPCR or ddPCR analysis.

9-10. (canceled)

11. A method for preparing a preparation of amplified DNA derived from a biological sample of a pregnant woman useful for non-invasive prenatal testing, comprising
(a) isolating cfDNA from a biological sample of a pregnant woman, wherein the isolated cfDNA comprises a mixture of fetal cfDNA and maternal cfDNA;
(b) preparing a preparation of amplified DNA by: optionally, ligating adaptors to the isolated cfDNA to obtain adaptor-ligated DNA, and/or amplifying the adaptor-ligated DNA to obtain amplified adaptor-ligated DNA;
(c) selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of fetal cfDNA; and
(d) performing a multiplex amplification reaction to amplify at least 100 polymorphic loci on the selectively enriched DNA in one reaction mixture; and
(c) analyzing the preparation of amplified DNA by determining the sequences of the selectively enriched DNA.

12. The method of claim 11, wherein the fraction of fetal cfDNA is increased by at least 20% in the selectively enriched DNA compared to the isolated cfDNA.

13. The method of claim 11, wherein the analyzing the preparation of amplified DNA further comprising determining the presence of at least one fetal chromosomal abnormality based on the sequences of the selectively enriched DNA, wherein the fetal chromosomal abnormality comprises single nucleotide variant (SNV), copy number variation (CNV), and/or chromosomal rearrangement.

14-18. (canceled)

19. The method of claim 11, wherein step-(d) the performing a multiplex amplification reaction comprises amplifying at least 1000 polymorphic loci on the selectively enriched DNA in one reaction mixture.

20. (canceled)

21. A method for preparing a preparation of amplified DNA derived from a biological sample of a transplant recipient useful for monitoring transplant rejection, comprising
(a) isolating cfDNA from a biological sample of a transplant recipient, wherein the isolated cfDNA comprises a mixture of donor-derived cfDNA and recipient cfDNA;
(b) preparing a preparation of amplified DNA by: optionally, ligating adaptors to the isolated cfDNA to obtain adaptor-ligated DNA, and/or amplifying the adaptor-ligated DNA to obtain amplified adaptor-ligated DNA;
selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of donor-derived cfDNA; and
performing a multiplex amplification reaction to amplify at least 100 polymorphic loci on the selectively enriched DNA in one reaction mixture; and
(c) analyzing the preparation of amplified DNA by determining the sequences of the selectively enriched DNA.

22. The method of claim 21, wherein the fraction of donor-derived cfDNA is increased by at least 20% in the selectively enriched DNA compared to the isolated cfDNA.

23. The method of claim 21, wherein the analyzing the preparation of amplified DNA further comprising quantifying the amount of donor-derived cfDNA.

24-29. (canceled)

30. The method of claim 21, wherein the method comprises longitudinally collecting one or more biological samples from the transplant recipient after transplantation, and repeating steps (a)-(c) for each biological samples longitudinally collected.

31. A method for preparing a preparation of amplified DNA derived from a biological sample of a subject diagnosed with cancer useful for monitoring relapse or metastasis of cancer, comprising
(a) isolating cfDNA from a biological sample of a subject diagnosed with cancer;
(b) preparing a preparation of amplified DNA by: optionally, ligating adaptors to the isolated cfDNA to obtain adaptor-ligated DNA, and/or amplifying the adaptor-ligated DNA to obtain amplified adaptor-ligated DNA;
selectively enriching trinucleosomal, dinucleosomal, mononucleosomal or sub-mononucleosomal DNA from the isolated cfDNA, the adaptor-ligated DNA or the amplified adaptor-ligated DNA to obtain selectively enriched DNA, wherein the selectively enriched DNA comprises an increased fraction of circulating tumor DNA (ctDNA); and
performing a multiplex amplification reaction to amplify a plurality of patient-specific somatic mutations on the selectively enriched DNA in one reaction mixture, wherein the patient-specific somatic mutations are identified in a tumor sample of the subject; and
(c) analyzing the preparation of amplified DNA by determining the sequences of the selectively enriched DNA.

32-33. (canceled)

34. The method of claim 31, wherein the fraction of ctDNA is increased by at least 20% in the selectively enriched DNA compared to the isolated cfDNA.

35. The method of claim 31, wherein the analyzing the preparation of amplified DNA further comprises detection of two or more patient-specific somatic mutations in the selectively enriched DNA which is indicative of relapse or metastasis of cancer, wherein the patient-specific somatic mutations comprise single nucleotide variant (SNV), copy number variation (CNV), and/or chromosomal rearrangement.

36-41. (canceled)

42. The method of claim 31, wherein the method comprises longitudinally collecting one or more biological samples from the subject after the patient has been treated with surgery, first-line chemotherapy, and/or adjuvant therapy, and repeating steps (a)-(c) for each biological samples longitudinally collected.

43-46. (canceled)
